(19) United States
(12) Patent Application Publication
(10) Pub. No.: US 2025/0163502 A1
(43) Pub. Date: May 22, 2025

(54) METHODS FOR PROCESSING RIBONUCLEIC ACIDS IN BIOLOGICAL SAMPLES

(71) Applicant: 10x Genomics, Inc., Pleasanton, CA (US)

(72) Inventors: Justin COSTA, Union City, CA (US); Yina LI, Mountain View, CA (US); Meiliana TJANDRA, Dublin, CA (US)

(21) Appl. No.: 18/955,449

(22) Filed: Nov. 21, 2024

Related U.S. Application Data
(60) Provisional application No. 63/681,762, filed on Aug. 9, 2024.
(60) Provisional application No. 63/602,321, filed on Nov. 22, 2023.

(51) Int. Cl. C12Q 1/6841

(52) U.S. Cl. CPC C12Q 1/6841

(57) ABSTRACT

The present disclosure relates in some aspects to methods and compositions for repairing and/or immobilizing ribonucleic acid analytes in biological samples, and more specifically fragmented ribonucleic acids. A workflow is provided for polishing and repairing fragmented ribonucleic acids in a biological sample, for improving nucleic acid quality for downstream applications, such as in situ, spatial array, or single-cell based analysis. Ribonucleic acid analytes may be tethered covalently or non-covalently to a matrix-forming agent, for example, matrix-forming agent that can form a three-dimensional polymerized matrix.

### CROSS-REFERENCE TO RELATED APPLICATIONS

This application claims priority to U.S. Provisional Patent Application No. 63/602,321, filed on Nov. 22, 2023 entitled “METHODS FOR TETHERING RIBONUCLEIC ACIDS IN BIOLOGICAL SAMPLES,” and to U.S. Provisional Patent Application No. 63/681,762, filed on Aug. 9, 2024 entitled “METHODS FOR PROCESSING RIBONUCLEIC ACIDS IN BIOLOGICAL SAMPLES,” all of which are herein incorporated by reference in their entireties for all purposes.

### FIELD

The present disclosure relates in some aspects to methods for analyzing biological samples, and more specifically, methods for analyzing fragmented RNA.

### BACKGROUND

Despite improvements in transcriptomic analysis, many nucleic acid analytes present in biological samples can be lost throughout sample preparation using standard protocols and reagents, e.g., permeabilization and de-crosslinking, to enable analysis and imaging, such as by fluorescence in situ hybridization. Although the extent of losses of these uncaptured nucleic acid analytes out of the RNA transcripts for a given sample remain unknown, the missed RNA analytes nonetheless represent substantial segment of the overall transcriptome that remains omitted by analysis and constitute a significant gap in our knowledge for characterizing disease states of tissue samples.

Existing treatments to capture such analytes typically rely upon hybridization of the target RNA with complementary nucleic acid probes and cross-linking to the probes. However, these methods may still suffer from loss of RNA analytes in sample preparation. In instances where limited sample treatment is performed in order to preserve RNA, such target analytes are preserved intact but may be blocked by proteins, ribosomes, etc., that are also present, and, thus, may require large quantities of probe materials to obtain signal as a result.

Thus, improved methods and techniques for sequencing ribonucleic acids, particularly fragmented ribonucleic acids, are needed. Provided herein are methods and compositions that address such and other needs.

### SUMMARY

In some aspects, the methods and kits of the present disclosure provide means to anchor (or immobilize) ribonucleic acids which otherwise might be removed or destroyed during sample preparation, including fragmented ribonucleic acids, to exogenous or endogenous molecules present in a biological sample prior to sample work-up, thereby enabling downstream analysis of the ribonucleic acid analytes. In some instances, RNA analytes are fragmented in biological samples such as formalin-fixed, paraffin embedded (FFPE) cell or tissue samples, limiting the ability to capture longer RNA sequences for downstream analysis. In some aspects, the methods and kits of the present disclosure provide means to repair fragmented ribonucleic acids in a biological sample for improved downstream analysis (e.g., by probe hybridization and/or by sequencing).

In some aspects, provided herein is a method for analyzing a biological sample, comprising: (a) providing a biological sample comprising a ribonucleic acid (RNA) comprising a 5′-phosphate group or a 5′-phosphate group modified with a leaving group; (b) contacting the biological sample with an attachment agent, wherein the attachment agent comprises (i) at least one reactive moiety capable of reacting with at least one 5′-phosphate group of the RNA or at least one 5′-phosphate group of the RNA modified with a leaving group and (ii) at least one attachment moiety capable of attaching covalently or noncovalently to a matrix-forming agent; (c) forming a covalent bond between the reactive moiety of the attachment agent and the RNA; (d) contacting the biological sample with the matrix-forming agent; and (e) forming a three-dimensional polymerized matrix from the matrix-forming agent, thereby embedding the biological sample and immobilizing the RNA in the three-dimensional polymerized matrix.

In any of the embodiments herein, before the providing in (a) the method can comprise reacting at least one RNA in the biological sample with a polynucleotide kinase to provide the RNA comprising a 5′-phosphate group, and optionally modifying the 5′-phosphate group with a leaving group to provide the RNA comprising a 5′-phosphate group modified with a leaving group. In any of the embodiments herein, the polynucleotide kinase can be a T4 Polynucleotide Kinase (T4 PNK) or a T7 Polynucleotide Kinase (T7-PNK).

In any of the embodiments herein, the ribonucleic acid can be a fragmented and/or fixed RNA in the biological sample.

In any of the embodiments herein, the RNA can be mRNA.

In any of the embodiments herein, the attachment agent can be a compound of

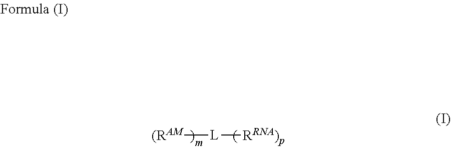

or a salt thereof, wherein: each R^{RNA} can be independently a reactive moiety capable of reacting with the at least one 5′-phosphate group of the RNA or the at least one 5′-phosphate group of the RNA modified with a leaving group; each R^{AM} can be independently an attachment moiety capable of attaching covalently or noncovalently to a matrix-forming agent; L can be a bond or a linker moiety; m can be an integer from 1 to 4; and p can be an integer from 1 to 4.

In any of the embodiments herein, the RNA provided in (a) can comprise a 5′-phosphate group.

In any of the embodiments herein, the at least one reactive moiety of the attachment agent can be capable of reacting with the at least one 5′-phosphate group of the RNA.

In any of the embodiments herein, the at least one reactive moiety of the attachment agent can comprise or can be a nucleic acid oligonucleotide comprising between 2 to 20 nucleotide residues. In any of the embodiments herein, the nucleic acid oligonucleotide can be a DNA oligonucleotide.

In any of the embodiments herein, the attachment agent can be of Formula (I-a):

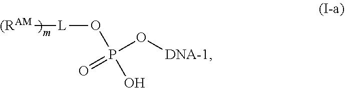

wherein: DNA-1 can comprise a nucleic acid sequence of 7-15 nucleotide residues; each R^{AM} can be independently an attachment moiety capable of attaching covalently or noncovalently to a matrix-forming agent; L can be a bond or a linker moiety; and m can be an integer from 1 to 4. In any of the embodiments herein, the DNA-1 can be at least one thymine (T). In any of the embodiments herein, the forming in (c) can comprise forming a covalent bond between a 3′-OH of the DNA-1 and a 5′-phosphate group of the RNA under the catalysis of a ligase. In any of the embodiments herein, the ligase can be T4 RNA Ligase 1.

In any of the embodiments herein, the RNA can comprise a 5′-phosphate group modified with a leaving group.

In any of the embodiments herein, the conjugation acid of the leaving group can have a pKa of less than 8.

In any of the embodiments herein, the leaving group can be any one of Cl, Br, I, —OR, —OC(O)R, —OS(O)₂R, or —NR¹R², wherein each R can be independently haloalkyl, phenyl substituted with one or more alkyl or haloalkyl, or heteroaryl substituted with one or more alkyl or haloalkyl, and wherein R¹ can be independently H, alkyl, or haloalkyl, R² can be independently haloalkyl, phenyl substituted with one or more alkyl or haloalkyl, or heteroaryl substituted with one or more alkyl or haloalkyl, or R¹ and R² can take together with the N atom to which they are attached to form a heteroaryl.

In any of the embodiments herein, the leaving group can be

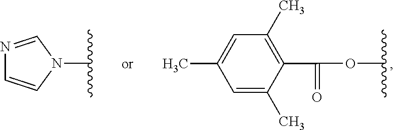 [SMILES: *n1ccnc1 ; *OC(=O)c1c(C)cc(C)cc1C]

wherein the wavy line can denote the attachment of the leaving group to a 5′ phosphate of the RNA.

In any of the embodiments herein, the forming in (c) can comprise forming a covalent bond between the reactive moiety of the attachment agent and a 5′-phosphate group of the RNA without catalysis of an enzyme.

In any of the embodiments herein, the reactive moiety can comprise or be a nucleophilic group.

In any of the embodiments herein, the reactive moiety can comprise or be —OH, —SH, or —NH₂.

In any of the embodiments herein, the attachment agent can be of Formula (I-b):

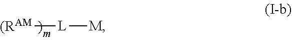

wherein M can be independently —NH₂, —OH, or —SH; each R^{AM} can be independently an attachment moiety capable of attaching covalently or noncovalently to a matrix-forming agent; L can be a bond or a linker moiety; and m can be an integer from 1 to 4.

In any of the embodiments herein, the attachment agent can comprise at least one attachment moiety capable of attaching covalently to a matrix-forming agent. In any of the embodiments herein, the at least one attachment moiety can be or can comprise an alkenyl, alkynyl, allyl or vinyl moiety, ally ester moiety, an acrylamide moiety, an amide moiety, an alcohol moiety, a polyol moiety, a furan moiety, a maleimide moiety, a norbornene moiety, a thiol moiety, a sulfide moiety, a phenol moiety, a urethane moiety, a cyano moiety, an amino moiety, an isocyanate moiety, an isothiocyanate moiety, an ether moiety, a dextran moiety, or an alginate moiety. In any of the embodiments herein, each attachment moiety of the at least one attachment moiety can independently be or can independently comprise a phenol moiety, an alkyne moiety, a norbornene moiety, a sulfide moiety, a furan moiety, a maleimide moiety, or an allyl ester moiety.

In any of the embodiments herein, the at least one attachment moiety can be or can comprise a moiety of Formula (I-b)

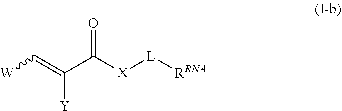

wherein R^{RNA} can be independently a reactive moiety capable of reacting with the at least one 5′-phosphate group of the RNA or the at least one 5′-phosphate group of the RNA modified with a leaving group; L can be a bond or a linker moiety; W can be independently H or C₁₋₆ alkyl; Y is H or C₁₋₆ alkyl; and X can be NH, N(C₁₋₆ alkyl), or O.

In any of the embodiments herein, the at least one attachment moiety can be or can comprise a click functional group. In any of the embodiments herein, the at least one attachment moiety can be or can comprise an azide moiety.

In any of the embodiments herein, the biological sample can be contacted with a labeling agent comprising a tethering moiety for attachment to the matrix.

In any of the embodiments herein, the attachment moiety can be capable of attaching non-covalently to the matrix-forming agent in the biological sample. In any of the embodiments herein, the attachment moiety can comprise or can be biotin.

In any of the embodiments herein, L can be unbranched or branched C₁-C₁₅₀ alkylene, optionally interrupted by 1 to 50 independently selected O, NH, N, S, C₆-C₁₂ arylene, or 5- to 12-membered heteroarylene. In any of the embodiments herein, L can be the group

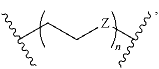

wherein Z can be CH₂, O, S, or NH; and n can be an integer from 0 to 50. In any of the embodiments herein, the biological sample embedded in the three-dimensional polymerized matrix can be cleared. In any of the embodiments herein, the biological sample can be cleared with a detergent, a lipase, and/or a protease. In any of the embodiments herein, the detergent can comprise a non-ionic surfactant or anionic surfactant, optionally wherein the detergent can comprise SDS, tergitol, NP-40, saponin, polyethylene glycol p-(1,1,3,3-tetramethylbutyl)-phenyl ether, or polysorbate 20, or any combinations thereof. In any of the embodiments herein, the protease can comprise proteinase K, pepsin, or collagenase, trypsin, dispase, thermolysin, or alpha-chymotrypsin, or any combinations thereof, optionally wherein the protease can comprise Liberase™. In any of the embodiments herein, the lipase can comprise a pancreatic, hepatic and/or lysosomal lipase, or any combinations thereof, optionally wherein the lipase can comprise sphingomyelinase or esterase, or a combination thereof.

In any of the embodiments herein, m can be 1.

In any of the embodiments herein, the biological sample can be contacted with a probe or probe set that binds directly or indirectly to the ribonucleic acid, optionally wherein the probe or probe set can be a detectable probe. In some embodiments, the probe or probe set is added to the biological sample after immobilizing the RNA in the three-dimensional polymerized matrix. In any of the embodiments herein, the probe or probe set can be a circular or circularizable probe or probe set, optionally wherein the method can comprise circularizing the circularizable probe or probe set using the ribonucleic acid or a product thereof as a template, optionally wherein the method can comprise generating an RCA product using the circular or circularizable probe as a template. In some embodiments, the immobilized RNA is used as a primer to perform RCA. In some embodiments, the RCA product is detected at the location of the immobilized RNA. In any of the embodiments herein, the biological sample can be imaged to detect the probe or probe set or the RCA product. In any of the embodiments herein, imaging can comprise detecting a signal associated with the probe or probe set or the RCA product, optionally wherein the signal can be from a fluorescently labeled probe that directly or indirectly binds to the probe or probe set or the RCA product. In any of the embodiments herein, the probe or probe set can comprise a barcode sequence. In any of the embodiments herein, the method can comprise detecting the barcode sequence or a complement thereof in the probe or probe set or in a product of the probe or probe set. In some examples, a sequence of the RCA product is detected by performing sequencing-by-synthesis (SBS), sequencing-by-avidity (SBA) or sequencing-by-binding (SBB). In some examples, a sequence of the RCA product is detected by performing sequential cycles of hybridization with a plurality of labelled probes. In some examples, one or more barcode sequence(s) in the RCA product is detected by performing sequential cycles of hybridization with a plurality of labelled probes. In some examples, one or more barcode sequence(s) in the RCA product is detected by performing sequencing-by-synthesis (SBS), sequencing-by-avidity (SBA) or sequencing-by-binding (SBB).

In any of the embodiments herein, the method can further comprise contacting the biological sample with an antibody attached to an oligonucleotide comprising a functional moiety for attachment to the matrix. In some embodiments, the method comprises attaching the functional moiety to the matrix. In some embodiments, method comprises clearing the biological sample after attaching the functional moiety to the matrix.

In any of the embodiments herein, the method can comprise covalently or non-covalently attaching the 3′ end of the RNA to the matrix. In some embodiments, the 3′ end of the RNA is attached to the matrix using an oligonucleotide comprising a plurality of thymine residues and a functional moiety for attachment to the matrix.

In any of the embodiments herein, the method can comprise treating the biological sample with a detergent and a protease after forming the three-dimensional polymerized matrix. In any of the embodiments herein, the detergent can comprise SDS and the protease can comprise proteinase K. In any of the embodiments herein, the detergent and the protease can be provided in a buffer of at least pH 8.0. In any of the embodiments herein, the biological sample can be treated with the detergent and the protease at at least 45° C. for no more than 4 minutes. In any of the embodiments herein, the biological sample can be treated with the detergent and the protease at about 50° C. for about 3 minutes. In any of the embodiments herein, the biological sample can be treated with 1% SDS and 200 μg/mL proteinase K provided in a PBS buffer of at least pH 8.5 at about 50° C. for about 3 minutes.

In some aspects, herein is provided a method for processing a biological sample, comprising: a) treating a cell or tissue sample with a polynucleotide kinase, wherein the polynucleotide kinase repairs a 3′ end of a first fragment of a cellular ribonucleic acid (RNA) in the cell or tissue sample and phosphorylates a 5′ end of a second fragment of the cellular RNA in the cell or tissue sample using a polynucleotide kinase, and b) after treating the cell or tissue sample with the polynucleotide kinase, ligating the 3′ end of the first fragment of the cellular RNA to the 5′ end of the second fragment of the cellular RNA to generate a ligated RNA at a location in the cell or tissue sample.

In some embodiments, the method further comprises immobilizing the ligated RNA by tethering the ligated RNA to the biological sample or to a matrix embedding the biological sample. In some embodiments, the ligated RNA is at least 200 nucleotides in length, optionally wherein the ligated RNA is at least 300 nucleotides in length. In some embodiments, the cellular RNA is a messenger RNA (mRNA). In some embodiments, the 3′ end of the second fragment of the cellular RNA is end-repaired and the 5′ end of a third fragment of the cellular RNA is phosphorylated, and the method comprises ligating the 3′ end of the second fragment of the cellular RNA to the 5′ end of the third fragment of the cellular RNA. In some embodiments, the polynucleotide kinase is a T4 Polynucleotide Kinase (T4 PNK) or a T7 Polynucleotide Kinase (T7-PNK).

In some embodiments, treating the biological sample with the polynucleotide kinase comprises incubating the sample with the polynucleotide kinase in a buffer comprising ATP and a divalent cation cofactor of the polynucleotide kinase. In some embodiments, treating the biological sample with the polynucleotide kinase comprises incubating the biological sample with the polynucleotide kinase in a buffer having a pH between about 7 and about 8, optionally wherein the buffer has a pH of about 7.6. In some embodiments, treating the biological sample with the polynucleotide kinase comprises incubating the biological sample with the polynucleotide kinase in an acidic buffer, optionally wherein the pH of the acidic buffer is about 6. In some embodiments, treating the biological sample with the polynucleotide kinase comprises (i) incubating the biological sample with the polynucleotide kinase, ATP, and a divalent cation cofactor of the polynucleotide kinase at a pH of between about 7 and 8, and (ii) incubating the biological sample with the polynucleotide kinase and the divalent cation cofactor at a pH of about 6, wherein (i) and (ii) are performed in either order. In some embodiments, (i) comprises incubating the biological sample in a first buffer comprising the polynucleotide kinase, ATP, and the divalent cation cofactor, and (ii) comprises incubating the biological sample with a second buffer comprising the polynucleotide kinase the divalent cation cofactor at a pH of about 6. In some embodiments, the second buffer does not comprise ATP.

In some embodiments, the ligating is performed using a T4 RNA ligase, optionally wherein the T4 RNA ligase is a T4 RNA ligase 1.

In some embodiments, the sample is embedded in a matrix, and the method further comprises clearing the biological sample embedded in the matrix. In some embodiments, the biological sample is cleared with a detergent, a lipase, and/or a protease. In some embodiments, the detergent comprises a non-ionic surfactant or anionic surfactant, optionally wherein the detergent comprises SDS, tergitol, NP-40, saponin, polyethylene glycol p-(1,1,3,3-tetramethylbutyl)-phenyl ether, or polysorbate 20, or any combinations thereof. In some embodiments, the protease comprises proteinase K, pepsin, or collagenase, trypsin, dispase, thermolysin, or alpha-chymotrypsin, or any combinations thereof, optionally wherein the protease comprises Liberase™. In some embodiments, the lipase comprises a pancreatic, hepatic and/or lysosomal lipase, or any combinations thereof. In some embodiments, the lipase comprises sphingomyelinase or esterase, or a combination thereof. In some embodiments, the method further comprises covalently or non-covalently attaching the ligated RNA to a matrix. In some embodiments, the first fragment of the cellular RNA and the second fragment of the cellular RNA are not attached to a matrix.

In some embodiments, the method comprises treating the biological sample with a detergent and a protease after forming a matrix embedding the biological sample. In some embodiments, the detergent comprises SDS and the protease comprises proteinase K. In some embodiments, the detergent and the protease are provided in a buffer of at least pH 8.0. In some embodiments, the biological sample is treated with the detergent and the protease at at least 45° C. for no more than 4 minutes. In some embodiments, the biological sample is treated with the detergent and the protease at about 50° C. for about 3 minutes. In some embodiments, the biological sample is treated with 1% SDS and 200 μg/mL proteinase K provided in a PBS buffer of at least pH 8.5 at about 50° C. for about 3 minutes.

In some embodiments, the method further comprises heating the biological sample or the ligated RNA to at least about 70° C. in a buffer having a pH of between about 4 and about 9. In some embodiments, the method further comprises contacting the biological sample or the ligated RNA with an organocatalyst capable of removing one or more formaldehyde adducts from RNA. In some embodiments, the organocatalyst comprises an anthranilate or a phosphanilate. In some embodiments, the biological sample is contacted with the organocatalyst after ligating the 3′ end of the first fragment of the cellular RNA to the 5′ end of the second fragment of the cellular RNA to generate the ligated RNA. In some embodiments, the method further comprises treating the biological sample or the ligated RNA with a glycosylase, optionally wherein the glycosylase excises a damaged base from the ligated RNA.

In some embodiments, the method further comprises performing reverse transcription of the ligated RNA to generate a cDNA product of the ligated RNA. In some embodiments, the reverse transcription is performed using a promiscuous or trans-lesion reverse transcriptase enzyme. In some embodiments, the reverse transcriptase is HIV-1 RT. In some embodiments, the reverse transcriptase is Murine Leukemia Virus RT. In some embodiments, the reverse transcription is performed in the presence of a nucleotide mixture comprising 5-methylcytosine and/or pseudouridine nucleotide analogs to the RT reaction.

In some embodiments, the method further comprises, after ligating the 3′ end of the first fragment of the cellular RNA to the 5′ end of the second fragment of the cellular RNA to generate the ligated RNA, contacting the biological sample with a probe or probe set that binds directly or indirectly to the ligated RNA or to the cDNA generated from the ligated RNA, optionally wherein the method comprises detecting the bound probe or probe set or a product thereof. In some embodiments, the probe or probe set is a circular or circularizable probe or probe set, optionally wherein the method comprises circularizing the circularizable probe or probe set using the ligated RNA or the cDNA generated from the ligated RNA as a template, optionally wherein the method comprises generating an RCA product using the circular or circularizable probe as a template. In some embodiments, the method comprises imaging the biological sample to detect the probe or probe set or the RCA product. In some embodiments, imaging the biological sample comprises detecting a signal associated with the probe or probe set or the RCA product, optionally wherein the signal is from a fluorescently labeled probe that directly or indirectly binds to the probe or probe set or the RCA product. In some embodiments, the probe or probe set comprises a barcode sequence that identifies the cellular RNA, optionally wherein the method comprises detecting the barcode sequence or a complement thereof in the probe or probe set or in a product of the probe or probe set.

In some embodiments, the method comprises capturing the ligated RNA on an array. In some embodiments, the method comprises sequencing all or a portion of the captured ligated RNA or a complement thereof, optionally wherein the captured ligated RNA or complement thereof is amplified. In some embodiments, the method comprises performing reverse transcription of the captured ligated RNA to generate a cDNA product of the captured ligated RNA on the array. In some embodiments, the reverse transcription is performed using a promiscuous or trans-lesion reverse transcriptase enzyme. In some embodiments, the reverse transcriptase is HIV-1 RT. In some embodiments, the reverse transcriptase is Murine Leukemia Virus RT. In some embodiments, the reverse transcription is performed in the presence of a nucleotide mixture comprising 5-methylcytosine and/or pseudouridine nucleotide analogs to the RT reaction.

In some embodiments, the method comprises sequencing all or a portion of the cDNA or a complement thereof, optionally wherein the cDNA or complement thereof is amplified. In some embodiments, the method comprises capturing the cDNA on an array. In some embodiments, the method comprises sequencing all or a portion of the captured cDNA or a complement thereof, optionally wherein the captured cDNA or complement thereof is amplified. In some embodiments, the method comprises generating an amplification product of the captured ligated RNA or complement thereof comprising a spatial barcode sequence or complement thereof that identifies the location of the captured ligated RNA on the array. In some embodiments, the method comprises generating an amplification product of the captured cDNA or complement thereof comprising a spatial barcode sequence or complement thereof that identifies the location of the captured cDNA on the array.

In some embodiments, the biological sample comprises a cell that is partitioned. In some embodiments, the partition is a microwell or a droplet. In some embodiments, the droplet is an emulsion droplet. In some embodiments, the partition comprises a support that comprises a plurality of barcode oligonucleotides comprising a partition barcode sequence and a capture sequence. In some embodiments, the barcode oligonucleotides are releasably attached to the support. In some embodiments, the support is a bead. In some embodiments, the bead is a gel bead. In some embodiments, the capture sequence binds to the ligated RNA or a product thereof. In some embodiments, using a barcode oligonucleotide of the plurality of barcode oligonucleotides to generate a barcoded cDNA product of the ligated RNA comprising a sequence of the partition barcode sequence or complement thereof. In some embodiments, generating the barcoded cDNA product comprises performing reverse transcription of the captured ligated RNA using a promiscuous or trans-lesion reverse transcriptase enzyme. In some embodiments, the reverse transcriptase is HIV-1 RT. In some embodiments, the reverse transcriptase is Murine Leukemia Virus RT. In some embodiments, the reverse transcription is performed in the presence of a nucleotide mixture comprising 5-methylcytosine and/or pseudouridine nucleotide analogs to the RT reaction.

In some embodiments, the method further comprises releasing the barcoded cDNA product or a complement thereof from the partition. In some embodiments, the method further comprises pooling the barcoded cDNA product or complement thereof from the partition with contents of other partitions of a plurality of partitions. In some embodiments, the method further comprises amplifying the barcoded cDNA product or complement thereof. In some embodiments, the method further comprises determining a sequence of the barcoded cDNA product or complement thereof.

In some embodiments, the biological sample is a tissue section. In some embodiments, the biological sample is a formalin-fixed sample. In some embodiments, the biological sample is a formalin-fixed, paraffin-embedded (FFPE) sample. In some embodiments, the biological sample is a deparaffinized FFPE sample.

In some aspects, provided herein is a system comprising a matrix-forming agent configured for forming a three-dimensional polymerized matrix from the matrix-forming agent to immobilize an RNA in the three-dimensional polymerized matrix; and an attachment agent, wherein the attachment agent comprises: (i) at least one reactive moiety capable of reacting with at least one 5′-phosphate group of an RNA or at least one 5′-phosphate group of an RNA modified with a leaving group, and (ii) at least one attachment moiety capable of attaching covalently or noncovalently to the matrix-forming agent.

In some embodiments, the matrix forming agent comprises one or more nucleophilic groups. In some embodiments, the one or more nucleophilic group is a sulfhydryl, hydroxyl, or amino functional group. In some embodiments, the matrix forming agent is acrylamide, bis-acrylamide, polyacrylamide or derivatives thereof, poly(ethylene glycol) or derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, poly acrylamide, poly(hydroxyethyl acrylate), poly(hydroxyethyl methacrylate), collagen, hyaluronic acid, chitosan, dextran, agarose, gelatin, alginate, a protein polymer, or methylcellulose.

In some embodiments, the attachment agent is a compound of Formula (I)

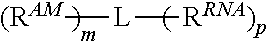

or a salt thereof, wherein: each R^{RNA} is independently a reactive moiety capable of reacting with the at least one 5′-phosphate group of the RNA or the at least one 5′-phosphate group of the RNA modified with a leaving group; each R^{AM} is independently an attachment moiety capable of attaching covalently or noncovalently to a matrix-forming agent; L is a bond or a linker moiety; m is an integer from 1 to 4; and p is an integer from 1 to 4.

In some embodiments, the system further comprises a clearing agent. In some embodiments, the clearing agent is a detergent, a lipase, and/or a protease.

In some embodiments, the system further comprises a probe or probe set comprising a region complementary to an RNA or to a probe that binds to the RNA. In some embodiments, the probe or probe set is a circular probe or circularizable probe or probe set.

In some embodiments, the system further comprises a ligase for ligating the probe or probe set.

In some embodiments, the system further comprises a polymerase for performing rolling circle amplification.

In some embodiments, the system further comprises a detectable probe comprising a region complementary to the probe or probe set, or a product thereof. In some embodiments, the detectable probe is a fluorescently-labeled probe.

In some embodiments, the system further comprises a sequencing primer complementary to a sequence comprised on the probe or probe set, or a product thereof.

In some embodiments, the system further comprises a nucleotide pool configured for performing a nucleic acid sequencing reaction.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings illustrate certain features and advantages of this disclosure. These embodiments are not intended to limit the scope of the appended claims in any manner.

FIG. 1 depicts an example of a method for enzymatically tethering the 5′ end of an RNA fragment comprising a 5′ phosphate using a ligase to attach DNA oligo containing 5′ attachment moiety (acrydite).

FIG. 2 depicts an example of a method according to the present disclosure for modifying ribonucleic acids with attachment agents via a non-enzymatic approach utilizing pyridine. In Step 1, RNA molecules containing 5′ phosphates are incubated with Carbonyldiimidizol and pyridine. In Step 2, 2-aminoethyl methacrylamide is added to the sample to yield hydrogel reactive RNA molecules.

FIGS. 3A and 3B depict example methods according to the present disclosure for modifying ribonucleic acids with attachment agents via non-enzymatic approaches that do not require pyridine. FIG. 3A depicts a method to incorporate 2-aminoethyl methacrylamide into ribonucleic acids at the 5′ position. FIG. 3B depicts a method to incorporate various attachment moieties (R) compatible with a variety of different hydrogel chemistries into ribonucleic acids at the 5′ position.

FIGS. 4A-4I depict results from an in situ analysis performed on mouse brain tissue sections using the 5′ tethering of RNA molecules disclosed herein. FIG. 4A depicts representative fluorescence microscopy images of control samples that were not processed to tether the 5′ ends of RNA molecules within the samples (control 1 and control 2), samples in which the 5′ ends of RNA molecules within the samples were enzymatically tethered according to the method of FIG. 1 (5′ Enzymatic Tether), and samples in which both the 5′ and 3′ ends of RNA molecules within the samples were enzymatically tethered (5′ 3′ Co-Tether). FIGS. 4B-4I depict the quantification of several parameters from fluorescent images of each tissue sample including the number of detectable blobs per channel (green, FIG. 4B; yellow, FIG. 4C; orange, FIG. 4D; and red, FIG. 4E), the number of decoded transcripts per 100 μm² (FIG. 4F), the density of detected objects per 100 μm² (FIG. 4G), the mean number of detected genes per cell (FIG. 4H), and the mean stripping error rate (FIG. 4I).

FIG. 5A depicts fragmentation of an RNA backbone via transesterification resulting in a 2′3′ cyclophosphate fragment and a 5′ hydroxyl fragment.

FIG. 5B depicts common 3′ end breaks which inhibit attempts at ligation and repair.

FIGS. 6A-6B depict enzymatic polishing (e.g., repairing) of common RNA fragments. FIG. 6A depicts enzymatic polishing of a 3′ RNA fragment with T4 PNK. FIG. 6B depicts enzymatic polishing of a 5′ RNA fragment with T4 PNK.

FIG. 7 depicts ligation of a 3′ fragment and a 5′ fragment to form a ligated RNA molecule.

FIG. 8 depicts an example workflow for improving RNA quality in a formalin-fixed, paraffin-embedded biological sample.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods, systems and kits for analyzing ribonucleic acids (RNA), e.g., highly degraded or fragmented RNA, in a biological sample (e.g., tissue). The methods, systems and kits provided herein can be applied to various applications such as in situ methods, spatial array capture and detection methods, and single-cell RNA sequencing methods. In situ analysis of the identity and spatial localization of RNA requires positional stability of the RNA. However, the preparation of many samples for in situ analysis undergo several harsh processing steps (e.g., formalin-fixed, paraffin-embedded (FFPE) tissues). These steps include baking and deparaffinization, decrosslinking, and permeabilization. The vast majority of RNA (e.g., mRNA) can be lost during and after the decrosslinking step. In some cases, FFPE samples for example have compromised RNA as degradation can occur in the obtaining, storage, and/or processing of the sample before fixation is performed. In FFPE samples, the greatest RNA degradation along the backbone has been shown to occur due to the paraffin wax and/or the subsequent wax removal steps.

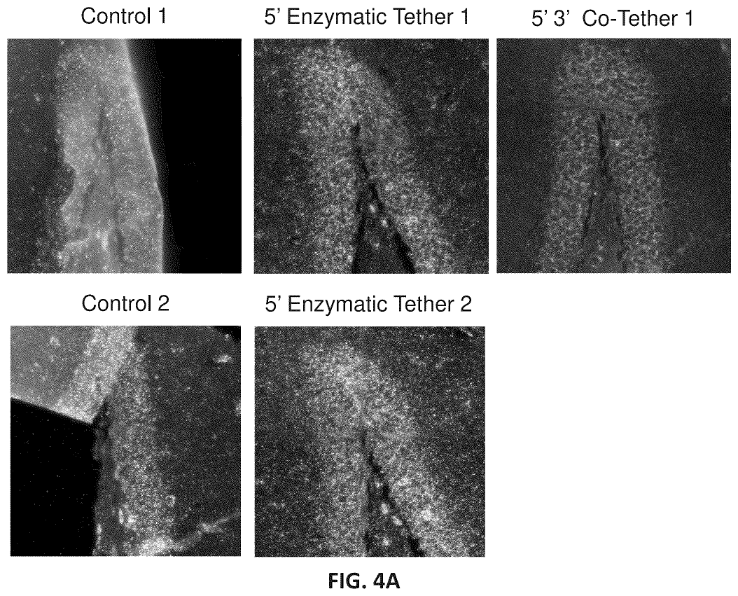

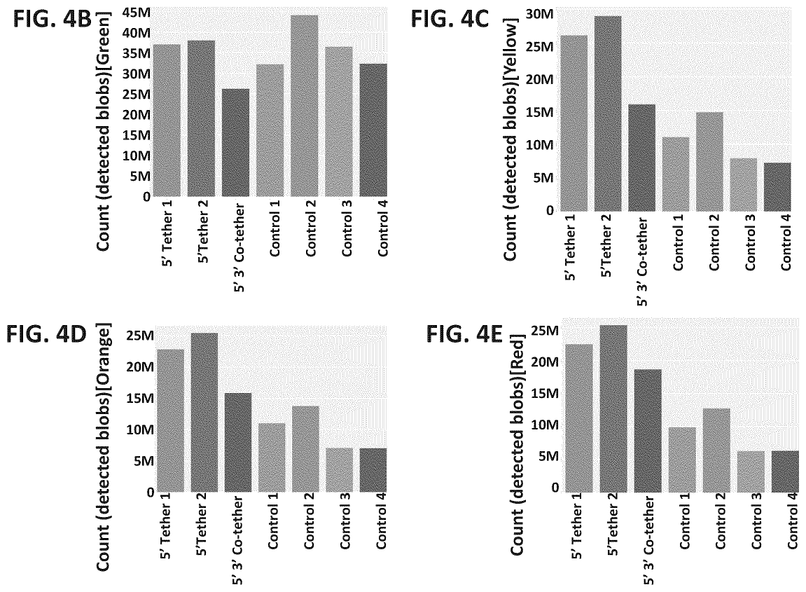

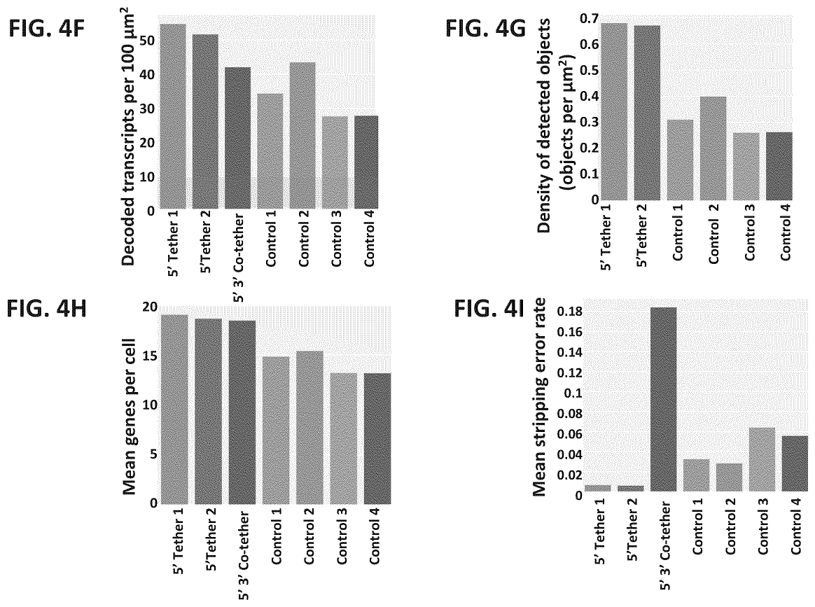

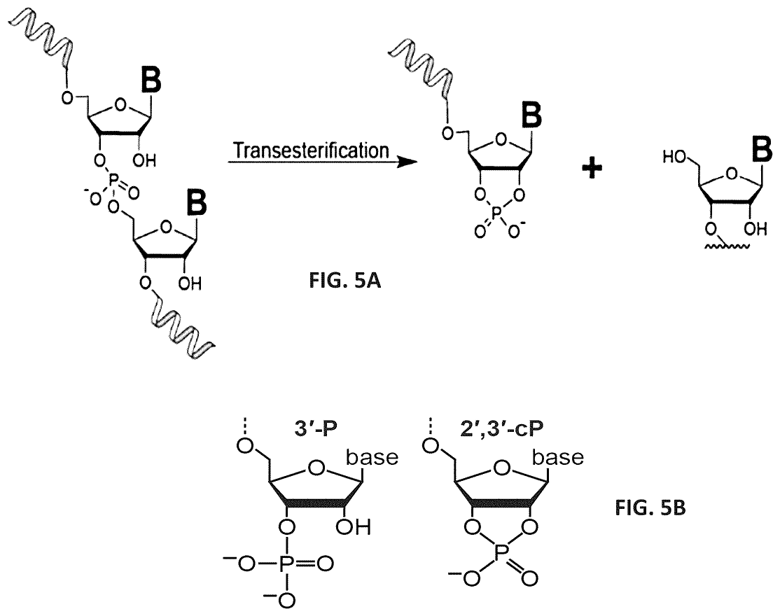

Hydrogel-based approaches to emerging in situ technologies offer certain advantages including reduced background autofluorescence and improved diffusional parameters, as well as enhanced tissue adhesion to the hydrogel and temporally spaced orthogonal chemistries that can be leveraged to couple tissue to the hydrogel. However, most hydrogel-based approaches have notable limitations. For example, pre-embedding a biological sample with hydrogel monomers prior to digestion of cellular components (e.g., proteins and lipids) and amplification is inherently more time consuming and limited in value for samples containing degraded or fragmented RNA. The methods and kits disclosed herein are intended to overcome many of these shortcomings through use of enzymatic or non-enzymatic reactions to link these biological samples (e.g., mRNA or fragmented RNA) with the hydrogel through an attachment agent described herein, particularly thorough 5′ end of the RNA in biological samples. Such 5′-tethering approaches allow all fragments to be spatially captured before decrosslinking, a step potentially leading to significant RNA loss. The approach provided herein also permits clearing of ribosomes from the mRNAs before introduction of probes (e.g., circularizable probes such as padlock probes), boosting sensitivity. For example, as shown in FIG. 4, 5′ enzymatic tethering of RNA molecules resulted in significant improvements in analyte detection compared to control tissue sections, including: a) an increase in the number of detected blobs observed in tissue samples in which RNA molecules were tethered via their 5′ ends (FIGS. 4B-4E); b) an increase in the number of decoded transcripts per 100 μm² (FIG. 4F); c) an increase in detected objects per μm² in 5′ tethered samples (FIG. 4G); and d) an increase in the number of genes detected per cell was improved in 5′ tethered samples compared to control samples (FIG. 4H). In some embodiments, tissue detachment from the substrate (e.g., glass slide) is minimized with the methods described herein to immobilize or tether ribonucleic acids. In some embodiments, the tissue sample is covalently attached to the substrate. Unlike common tethering approaches, which focus on capturing the 3′ end of RNA (e.g., using an oligo-dT modified with an acrydite), the methods provided herein focus on tethering 5′ end of RNA, allowing the 3′ end of the RNA to remain free for other applications such as priming an extension reaction using a 3′ end of the RNA (e.g., target-primed RCA). Therefore, the methods provided herein can enable pre-embedding approaches to samples that include degraded RNA while allowing RNA primed amplification, preserving their spatial orientation and opening access to thousands of RNA fragments/cell that have been unavailable to study until now. Additionally or alternatively, the methods provided herein allow for tethering RNA fragments at both the 3′ end and the 5′ end.

In some aspects, provided herein is a workflow for enzymatically repairing RNA fragments in a biological sample. Damage to RNA in fixed tissue samples, such as formalin-fixed, paraffin-embedded (FFPE) samples, limits RNA yield for downstream analysis, such as for in situ, single-cell, or spatial array-based methodologies. In some aspects, provided herein are methods for repair of RNA molecules, such as mRNA molecules, in a biological sample such as a FFPE cell sample or tissue section. In some embodiments, a workflow provided herein comprises enzymatically repairing or “polishing” 3′ ends of RNA fragments into 2′3′ vicinal diols that can then be ligated to 5′ ends of RNA fragments that have also undergone enzymatic polishing. In some embodiments, the polished (e.g., end repaired) RNA fragments are tethered covalently or noncovalently in the biological sample. In some embodiments, ribosomes are cleared from the biological sample to allow increased access to the RNA fragments. In some embodiments, nucleotide base adducts in the RNA fragments are repaired and/or excised. In some embodiments, RNA fragments are ligated together to generate repaired RNA molecules in the biological sample. In some embodiments, the repaired RNA molecules are reverse-transcribed for analysis in downstream applications.

### II. Methods for Tethering RNA

In some embodiments, the methods, systems and kits of the present disclosure provide means to anchor (or immobilize) ribonucleic acids which otherwise might be removed or destroyed during sample preparation. Also provided herein are methods for analyzing RNA in a biological sample (e.g., a tissue sample) (e.g., wherein the RNA is immobilized according to any of the methods disclosed herein). In some embodiments, the methods provided herein include a series of enzymatic and non-enzymatic reactions that are utilized to immobilize or tether any fragmented ribonucleic acids to an endogenous molecule in the biological sample or an exogenous molecule delivered to the biological sample, such as a matrix-forming agent. In some embodiments, the biological sample is a formalin-fixed paraffin-embedded (FFPE) tissue section. In some embodiments, the biological sample is a cell or tissue sample.

In some embodiments, the methods, systems and kits as provided herein enable tethering the 5′ end of RNA to a three-dimensional matrix, such as hydrogel. In some embodiments, ribonucleic acid having a fragmented terminal 5′ end is converted to RNA comprising a 5′-phosphate group. In some embodiments, the 5′-phosphate group moiety is further be linked to an attachment agent that allows the RNA to bond covalently or bind non-covalently to a matrix-forming agent. In some embodiments, the converted RNA is not directly labeled with a fluorophore.

In one aspect, provided herein is a method, comprising: (a) providing a biological sample comprising a ribonucleic acid (RNA) comprising a 5′-phosphate group or a 5′-phosphate group modified with a leaving group; (b) contacting the biological sample with an attachment agent; and (c) forming a covalent bond between the reactive moiety of the attachment agent and the RNA. In some embodiments, the method further comprises (d) contacting the biological sample with a matrix-forming agent. In some embodiments, (d) contacting the biological sample with the matrix is before the providing in (a) or after the forming in (c). In some embodiments, the contacting of (d) is after the forming of (c). In some embodiments, the method further comprises (e) forming a three-dimensional polymerized matrix from the matrix-forming agent, thereby embedding the biological sample and immobilizing the RNA in the three-dimensional polymerized matrix. In some embodiments, the forming of (e) is after the contacting (d), and the contacting of (d) is after the forming of (c). In some embodiments, the biological sample is a formalin-fixed paraffin-embedded (FFPE) tissue section.

In another aspect, provided herein is a method for analyzing a biological sample, the method comprising: (a) providing a biological sample comprising a ribonucleic acid (RNA) comprising a 5′-phosphate group or a 5′-phosphate group modified with a leaving group; (b) contacting the biological sample with an attachment agent; (c) forming a covalent bond between the reactive moiety of the attachment agent and the RNA; (d) contacting the biological sample with a matrix-forming agent; and (e) forming a three-dimensional polymerized matrix from the matrix-forming agent, thereby embedding the biological sample and immobilizing the RNA in the three-dimensional polymerized matrix. In some embodiments, the biological sample is a formalin-fixed paraffin-embedded (FFPE) tissue section.

In some aspects, provided herein is a method of analyzing a biological sample comprising a fragmented ribonucleic acid (RNA), the method comprising: (a) contacting the biological sample comprising the fragmented RNA with a polynucleotide kinase (optionally wherein the polynucleotide kinase is a T4 polynucleotides kinase), wherein the polynucleotide kinase catalyzes formation of a 5′-phosphate on the fragmented RNA; (b) contacting the biological sample with an attachment agent, wherein the attachment agent comprises (i) at least one reactive moiety capable of reacting with at least one 5′-phosphate group of the RNA or 5′-phosphate group of the RNA modified with a leaving group and (ii) at least one attachment moiety capable of attaching covalently or noncovalently to a matrix-forming agent, (c) forming a covalent bond between the reactive moiety of the attachment agent and the RNA; (d) contacting the biological sample with a matrix-forming agent; (e) forming a three-dimensional polymerized matrix from the matrix-forming agent, thereby embedding the biological sample in the three-dimensional polymerized matrix and anchoring the fragmented RNA to the three-dimensional polymerized matrix; (f) contacting the biological sample with a detergent and a protease; (g) contacting the biological sample with a probe or probe set that binds directly or indirectly to the RNA; and (h) detecting the probe or a product thereof at a location in the matrix. In some instances, the detergent and protease are provided in a buffer of at least pH 8.0 (optionally a buffer of at least pH 8.5). In some instances, the detergent and protease are contacted with the sample for no more than 4 minutes at at least 50° C. In some embodiments, the “product thereof” in reference to “the probe or a product thereof” is an amplification product of the probe or probe set. In some embodiments, the product thereof is a rolling circle amplification product of the probe or probe set. For example, the probe or probe set can be a circular probe, and the product thereof can be a rolling circle amplification product thereof. In another embodiment, the probe or probe set is circularized in the biological sample (e.g., by ligation) and the product thereof is a rolling circle amplification product of the circularized probe or probe set. In some embodiments, the detergent comprises SDS and the protease comprises proteinase K. In some embodiments, the biological sample is treated with 1% SDS and 200 μg/mL proteinase K provided in a PBS buffer of at least pH 8.5 at about 50° C. for about 3 minutes. In some embodiments, the biological sample is a formalin-fixed paraffin-embedded (FFPE) tissue section.

### A. Ribonucleic Acid(s)

In some embodiments, the RNA analyzed by a method provided herein comprises various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. In some embodiments, the RNA analyte is a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. In some embodiments, the RNA is small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). In some embodiments, the RNA is double-stranded RNA or single-stranded RNA. In some embodiments, the RNA is circular RNA. In some embodiments, the RNA is a bacterial rRNA (e.g., 16s rRNA or 23s rRNA).

As detailed above, the methods of the present disclosure are especially suitable for immobilization of fragmented RNA. In some embodiments, any one of the RNA analytes disclosed herein are fragmented. In some embodiments, the RNA analyzed by the methods provided herein comprises a fragmented RNA. In some embodiments, the RNA is a fragmented RNA. In some embodiments, the fragmented RNA is fragmented mRNA. In some embodiments, the fragmented RNA comprises a 5′-phosphate group or a 5′-phosphate group modified with a leaving group. In some embodiments, the 5′-phosphate group or 5′-phosphate group modified with a leaving group is a fragmented 5′ end of the RNA. In some embodiments, the 5′-phosphate group or 5′-phosphate group modified with a leaving group is a fragmented 5′ end of the RNA.

In some embodiments, before providing the RNA (e.g., in (a)), the method further comprises converting the 5′ end group of least one RNA in the biological sample into a 5′-phosphate group. In some embodiments, the method further comprises reacting at least one RNA in the biological sample with a polynucleotide kinase to provide the RNA comprising a 5′-phosphate group. In some embodiments, the polynucleotide kinase comprises a T4 Polynucleotide Kinase (T4 PNK). In some embodiments, the polynucleotide kinase comprises a T7 Polynucleotide Kinase (T7-PNK). In some embodiments, the polynucleotide kinase is a T4 PNK or a T7-PNK. In some embodiments, the polynucleotide kinase is T4 PNK. In some embodiments, the polynucleotide kinase is T7 PNK. In some embodiments, the biological sample is a formalin-fixed paraffin-embedded (FFPE) tissue section. In some embodiments, the biological sample comprises fragmented RNA. In some embodiments, the method comprises converting the 5′ end groups (e.g., 5′-OH of fragmented RNAs) of least a plurality of RNAs in the biological sample into 5′-phosphate groups.

In some embodiments, the resulting 5′-phosphate group is further modified with a leaving group, and thereby forming an RNA comprising a 5′-phosphate group modified with a leaving group. In some embodiments, the modification comprises replacing one —OH group in the 5′-phosphate group with a leaving group. In some embodiments, the conjugation acid of the leaving group has a pKa of less than 8, such as less than about any of 7.5, 7, 6.5, 6, 5.5, 5, 4.5, 4, 3.5, or 3. In some embodiments, the leaving group is any one of Cl, Br, I, —OR, —OC(O)R, —OS(O)₂R, or —NR¹R², wherein each R is independently haloalkyl, phenyl substituted with one or more alkyl or haloalkyl, or heteroaryl substituted with one or more alkyl or haloalkyl, and wherein R¹ is independently H, alkyl, or haloalkyl, R² is independently haloalkyl, phenyl substituted with one or more alkyl or haloalkyl, or heteroaryl substituted with one or more alkyl or haloalkyl, or R¹ and R² are taken together the N atom to which they are attached to form a heteroaryl. In some embodiments, the leaving group is

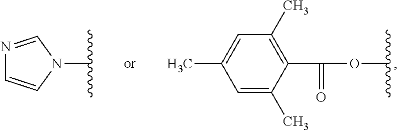 [SMILES: *n1ccnc1 ; *OC(=O)c1c(C)cc(C)cc1C]

wherein the wavy line denotes the attachment of the leaving group to the 5′ phosphate of the RNA.

In some embodiments, an RNA molecule lacking a 5′ phosphate is polished to form a 5′-phosphate group (e.g., on a fragmented 5′ end of the ribonucleic acid). In some embodiments as provided herein, the methods of the present disclosure utilize enzymatic reactions, driven by polynucleotide kinase, to convert RNA fragments lacking 5′-phosphate group into a RNA comprising a 5′-phosphate group. In some embodiments, the RNA comprising a 5′-phosphate group is generated from a fragmented RNA having 5′-OH fragmentation at the 5′-terminal end. In some embodiments, the biological sample is a formalin-fixed paraffin-embedded (FFPE) tissue section.

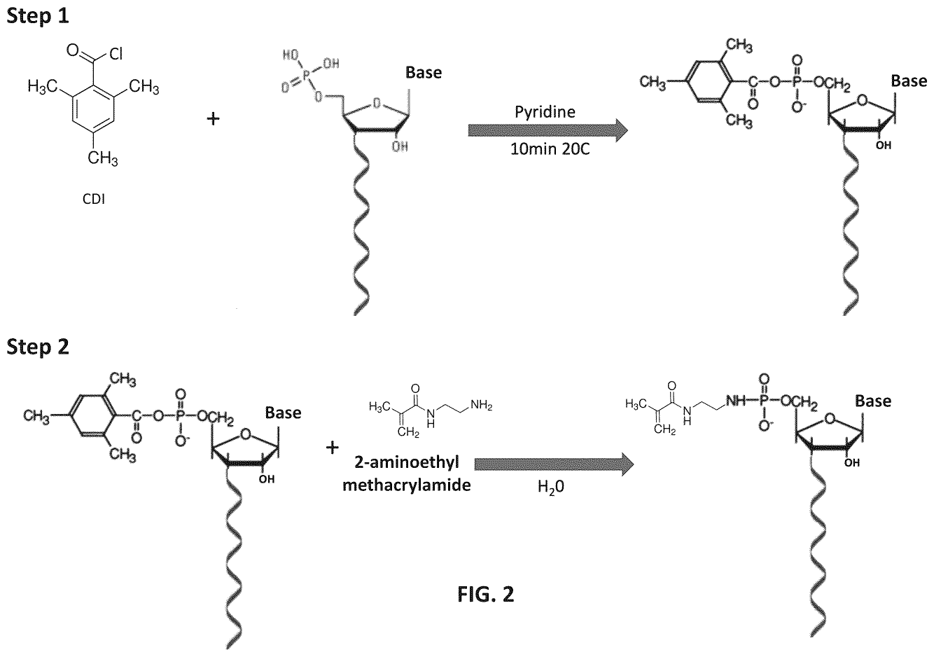

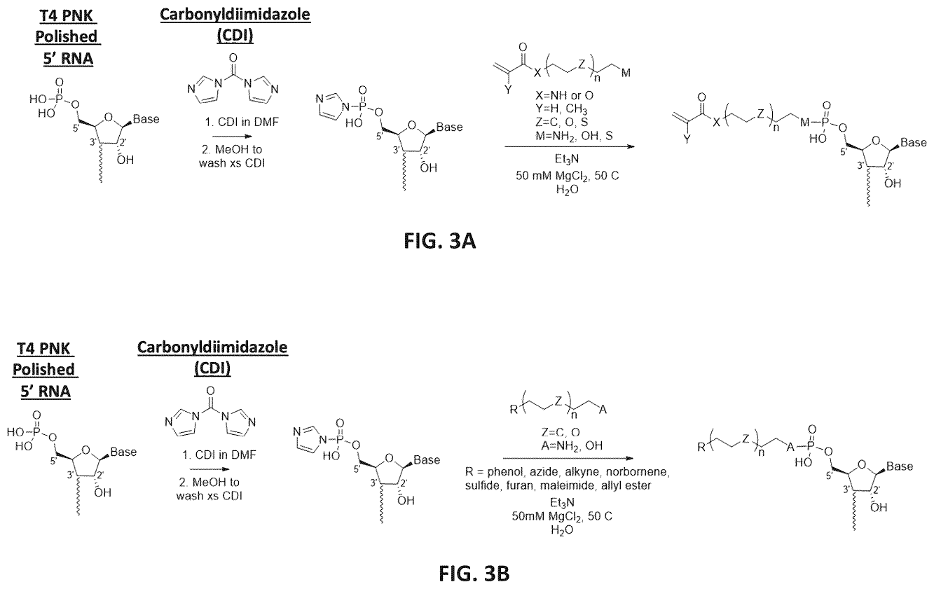

In FIG. 2 and FIG. 3A, various embodiments of mechanisms for modifying 5′ phosphate of the RNA are shown. In FIG. 2, the modification comprises reacting the 5′-phosphate group with carbonyldiimidazole (CDI) and thereby forming a 5′ phosphate modified with

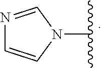 [SMILES: *n1ccnc1]

In FIG. 3A, the modification comprises reacting the 5′-phosphate group with 2,4,6-trimethylbenzoyl chloride and thereby forming a 5′ phosphate modified with

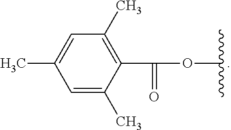 [SMILES: *OC(=O)c1c(C)cc(C)cc1C]

In some embodiments, the method comprises incubating the biological sample comprising the RNA in 100 mM Carbonyldiimidizol in anhydrous Dimethyl formamide. In some embodiments, the method comprises incubating the biological sample comprising the RNA in 100 mM Carbonyldiimidizol in anhydrous Dimethyl formamide for about 2 hours at a temperature between 18° C. and 22° C., optionally wherein the temperature is about 20° C. In some embodiments, after the incubation with Carbonyldiimidizol in anhydrous Dimethyl formamide, the method comprises a 10 minute 20° C. incubation in Pyridine. In some embodiments, after the Pyridine incubation, the method comprises incubating the biological sample with 2-aminoethyl methacrylamide (2-AEM) in H2O to generate modified hydrogel reactive RNA molecules in the biological sample. In some embodiments, the method comprises contacting the biological sample with a matrix-forming agent. In some embodiments, the method comprises forming a three-dimensional polymerized matrix from the matrix-forming agent, thereby embedding the biological sample. In some embodiments, the method comprises immobilizing the RNA in the three-dimensional polymerized matrix. In some embodiments, the method comprises incubating the biological sample with a polynucleotide kinase (e.g., T4 PNK) and a nucleotide triphosphate prior to incubating the biological sample in the Carbonyldiimidizol in anhydrous Dimethyl formamide.

In some embodiments, the method comprises incubating the biological sample comprising the RNA in 100 mM Carbonyldiimidizol in anhydrous Dimethyl formamide. In some embodiments, the method comprises incubating the biological sample comprising the RNA in 100 mM Carbonyldiimidizol in anhydrous Dimethyl formamide for about 2 hours at a temperature between 18° C. and 22° C., optionally wherein the temperature is about 20° C. In some embodiments, after the incubation with Carbonyldiimidizol in anhydrous Dimethyl formamide, the method comprises washing the biological sample in MeOH (e.g., performing 3 washes in MeOH). In some embodiments, after the MeOH washes, the method comprises incubating the biological sample in 2-AEM, MgCl₂, Triethylamine, Aniline and RNAse inhibitor. In some embodiments, after the MeOH washes, the method comprises a 30-minute 50° C. incubation in 100 mM 2-AEM, 30 mM MgCl₂, 100 mM Triethylamine, 1 nM Aniline and RNAse inhibitor. In some embodiments, the method comprises contacting the biological sample with a matrix-forming agent. In some embodiments, the method comprises forming a three-dimensional polymerized matrix from the matrix-forming agent, thereby embedding the biological sample. In some embodiments, the method comprises immobilizing the RNA in the three-dimensional polymerized matrix. In some embodiments, the method comprises incubating the biological sample with a polynucleotide kinase (e.g., T4 PNK) and a nucleotide triphosphate prior to incubating the biological sample in the Carbonyldiimidizol in anhydrous Dimethyl formamide.

The methods of the present disclosure may also be extended to direct immobilization of any RNA analytes, whether fragmented or not, wherein the RNA analyte possesses a 5′-phosphate group. In some embodiments, the RNA is not fragmented RNA.

In some embodiments of the methods as described herein, the method further comprises contacting or treating the biological sample and/or RNA with RNase inhibitors to prevent any undesired fragmentation. In some embodiments, the method further comprises the biological sample and fragmented ribonucleic acid are treated with a ribonuclease inhibitor. In some embodiments, the biological sample is contacted with a degradation agent to induce fragmentation, optionally wherein the method further comprises treating the biological sample and fragmented ribonucleic acid with a ribonuclease inhibitor after being contacted with the degradation agent. In some embodiments, the biological sample and fragmented ribonucleic acid are treated with a ribonuclease inhibitor after the biological sample has been contacted with a degradation agent. In some embodiments, the biological sample is treated with one or more RNase inhibitors. In some embodiments, the one or more RNase inhibitors are the same. In some embodiments, the one or more RNase inhibitors are different.

Examples of ribonuclease inhibitors include but are not limited to an anti-RNase antibodies, recombinant enzymes, or non-enzymatic inhibitors.

In some embodiments, the anti-RNase antibody is capable of binding to RNase A, RNase B, RNase C, RNase E, RNase H, RNase HI, RNase HII, RNase II, RNase III, RNase F1, RNase L, RNase M, RNase Ms, RNase N, RNase P, RNase PhyM, RNase R, RNase Sa, RNase St, RNase T1, RNase T2, RNase U2, RNase IV, RNase V, RNase E, RNase E, polynucleotide phosphorylase (PNPase), RNase PH, RNase, RNase BN, RNase D, RNase T, RNase 1, oligoribonuclease, exoribonuclease I, or exoribonuclease II. In some embodiments, the anti-RNase antibody comprises the Roche Protector RNase inhibitor (Millipore Sigma, Cat. #C756R82), In some embodiments, the anti-RNase antibody is Roche Protector RNase inhibitor.

In some embodiments, the recombinant enzyme is capable of degrading RNase A, RNase B, RNase C, RNase E, RNase H, RNase HI, RNase HII, RNase II, RNase III, RNase F1, RNase L, RNase M, RNase Ms, RNase N, RNase P, RNase PhyM, RNase R, RNase Sa, RNase St, RNase T1, RNase T2, RNase U2, RNase IV, RNase V, RNase E, RNase E, polynucleotide phosphorylase (PNPase), RNase PH, RNase, RNase BN, RNase D, RNase T, RNase 1, oligoribonuclease, exoribonuclease I, or exoribonuclease II. In some embodiments, the recombinant enzyme comprises Invitrogen's SUPERaseIn™ RNase Inhibitor, RNaseOUT™ Recombinant Ribonuclease Inhibitor, RNAsecure™ RNase Inactivation Reagent, or Ambion™ RNase Inhibitor.

In some embodiments, the non-enzymatic ribonuclease inhibitor comprises a ribonucleotide-derived RNase inhibitor or a nonnucleotide RNase inhibitor. In some embodiments, the RNase inhibitor is a vanadium salt. In some embodiments, the ribonucleotide-derived RNase inhibitor comprises one or more ribonucleotide vanadyl complexes (RVC), dinucleotide derivatives of adenosine 5′-pyrophosphate, such as 5′-diphosphoadenosine 3′-phosphate (ppA-3′-p) or 5′-diphosphoadenosine 2′-phosphate (ppA-2′-p), diadenosine derivatives, 3′-N-alkylamino-3′-deoxy-ara-uridines, or ribonucleotide zinc complexes, such as 3′-N-oxyurea-3′-deoxythymidine 5′-phosphate zinc complex. In some embodiments, the nonnucleotide RNase inhibitor comprises 8-amino-5-(4′-hydroxybiphenyl-4-ylazo)naphthalene-2-sulfonate or a catechin, such as epi-gallocatechin-3-gallate.

In some embodiments, the ribonucleic acid is a fragmented and/or fixed RNA in the biological sample. In some embodiments, the ribonucleic acid is a fragmented RNA in the biological sample. In some embodiments, the RNA is mRNA.

### B. Polynucleotide Kinase

As detailed above, the methods of the present disclosure employ enzymatic conversion of an RNA lacking 5′-phosphate (e.g., a fragmented RNA) into a RNA comprising a 5′-phosphate group. In some embodiments, the 5′ phosphate group is subsequently modified to enable downstream chemistries and immobilization in the biological sample. In some embodiments, the RNA lacking a 5′-phosphate is an endogenous RNA that is fragmented in the biological sample, resulting in an RNA comprising a 5′-OH group.

In some embodiments, the methods of the present disclosure comprise contacting the biological sample with a polynucleotide kinase for performing a 5′-end repairing reaction (also known as polishing) to phosphorylate a 5′-OH of an RNA in the biological sample. Polynucleotide kinase (PNK) enzymes are present in diverse bacterial taxa An example listing of PNKs is provided in the SIB Swiss Institute of Bioinformatics Expasy enzyme nomenclature database (entry: EC 2.7.1.78). In some embodiments, the PNK comprises a T4 polynucleotide kinase. In some embodiments, the PNK is T4 polynucleotide kinase.

In some embodiments, the PNK is contacted with the biological sample in the presence of magnesium. In some embodiments, the PNK comprises the N-terminal Pnk domain of T4 PNK. In some embodiments, the PNK is a T4 PNK or a homolog thereof. In some embodiments, the PNK is a T7 PNK or a homolog thereof. In some embodiments, the PNK is a Runella slithyformis HD-Pnk or a homolog thereof.!

In some embodiments, a biological sample comprising fragmented ribonucleic acids comprising a 5′-OH fragmentation at the 5′-terminal is treated with a 5′ kinase, such as T4 polynucleotide kinase, thereby resulting in the formation of a 5′-phosphate moiety at the 5′-terminal ribose ring. In some embodiments, the 5′-phosphate at the 5′ end of the fragmented RNA is generated by a PNK. In some embodiments, the 5′-phosphate is provided by contacting the fragmented ribonucleic acid with a 5′ kinase. In some embodiments, the fragmented ribonucleic acid has a 5′-OH, wherein the 5′ kinase catalyzes the formation of the 5′-phosphate.

In some embodiments, the method comprises treating the biological sample with T4 polynucleotide kinase (T4 PNK) for about between about 15 minutes and about 1 hour, between about 30 minutes and about 1 hour, between about 30 minutes and 2 hours, between about 1 hour and 2 hours, between about 20 minutes and 2 hours, or between about 20 minutes and 1 hour, at about 30° C. to about 45° C., or about 35° C. to about 40° C. (e.g., at about 37° C.). In some embodiments, the method comprises treating the biological sample with T4 polynucleotide kinase (T4 PNK) for about 1 hour at about 37° C. In some embodiments, the method comprises treating the biological sample with T4 polynucleotide kinase (T4 PNK) for about 30 minutes at about 37° C. In some embodiments, the biological sample is contacted with the polynucleotide kinase (PNK) and a nucleotide triphosphate (e.g., ATP). In some embodiments, the biological sample is contacted with the PNK and between any one of about 0.1-10, 0.5-5, 0.5-2, 0.7-1, or 1-2 mM ATP to polish the 5′ and 3′ ends of the RNA in situ. In some embodiments, the biological sample is contacted with the PNK and about 1 mM ATP to polish the 5′ and 3′ ends of the RNA in situ.

### C. Attachment Agent

As detailed herein, the methods of the present disclosure encompass the preparation of a ribonucleic acid (RNA) comprising a 5′-phosphate group or a 5′-phosphate group modified with a leaving group for immobilization of the ribonucleic acids in a matrix. The methods provided herein achieve immobilization of the ribonucleic acids through the use of an attachment agent that mediates the interaction between the ribonucleic acid and the matrix-forming agent and ultimately the matrix. In some embodiments, the methods as provided herein comprise contacting the biological sample with one or more attachment agent. In some embodiments wherein the method comprises contacting the biological sample with two or more attachment agents, the attachment agents is the same or different.

In other embodiments, the attachment agent is a multifunctional molecule. In some embodiments, the attachment agent comprises at least one (e.g., 1, 2, 3, or 4) reactive moiety capable of covalently bonding to the ribonucleic acid and at least one (e.g., 1, 2, 3, or 4) attachment moiety capable of covalently or non-covalently bonding to a matrix-forming agent. It should be recognized that reference to the attachment agent, whether bi- or multifunctional, as defined herein refers to the attachment agent prior to binding with the ribonucleic acid and the matrix-forming agent, unless otherwise noted.

In some embodiments, the attachment agent is a compound of Formula (I):

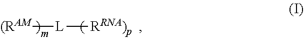

or a salt thereof, wherein each R^{RNA} is independently a reactive moiety capable of reacting with at least one 5′-phosphate group of the RNA or 5′-phosphate group of the RNA modified with a leaving group; each R^{AM} is independently an attachment moiety capable of attaching covalently or noncovalently to a matrix-forming agent; L is a bond or a linker moiety; m is an integer from 1 to 4; and p is an integer from 1 to 4.

In some embodiments, R^{RNA}, R^{AM}, L, m, and p are each as defined herein. It should be understood that every description, variation, embodiment or aspect of a moiety may be combined with every description, variation, embodiment or aspect of other moieties the same as if each and every combination of descriptions is specifically and individually listed. For example, every description, variation, embodiment or aspect provided herein with respect to R^{RNA} of Formula (I) may be combined with every description, variation, embodiment or aspect of L of Formula (I) the same as if each and every combination were specifically and individually listed. For another example, every description, variation, embodiment or aspect provided herein with respect to R^{AM} of Formula (I) may be combined with every description, variation, embodiment or aspect of L of Formula (I) the same as if each and every combination were specifically and individually listed.

i. Reactive moiety R^{RNA}

As provided herein, the reactive moiety capable of covalently bonding to the ribonucleic acid can be any reactive moiety that reacts with and covalently bonds to a 5′-phosphate or a 5′-phosphate modified with a leaving group. In some variations, such reactive moiety capable of covalently bonding to the ribonucleic acid by covalently bonding to a 5′-phosphate on the ribonucleic acid. In some variations, such reactive moiety capable of covalently bonding to the ribonucleic acid by covalently bonding to a 5′-phosphate on the ribonucleic acid modified with a leaving group.

In some embodiments, at least one reactive moiety is capable of reacting with at least one 5′-phosphate group of the RNA via an enzymatic reaction. In some embodiments, at least one reactive moiety of the attachment agent comprises or is a nucleic acid oligonucleotide comprising between 2 to 30 (e.g., between any of 2 to 25, 2 to 20, 2 to 15, or 5 to 15) nucleotide residues. In some embodiments, the nucleic acid oligonucleotide is a DNA oligonucleotide. The nucleic acid oligonucleotide can comprise any nucleic acid sequence, (e.g., any sequence of nucleotide residues). In some embodiments, the nucleic acid oligonucleotide comprises a random sequence. In some embodiments, the nucleic acid oligonucleotide comprises at least one thymine (T). In some embodiments, the nucleic acid oligonucleotide comprises a sequence of at least 2, 3, 4, 5, 6, 7, 8, or more thymines. In some embodiments, the nucleic acid oligonucleotide does not comprise a sequence of thymines. In some embodiments, p is 4. In some embodiments, p is 3. In some embodiments, p is 2. In some embodiments, p is 1. In some embodiments, the attachment agent is of Formula (I-a):

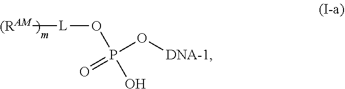

wherein DNA-1 comprises a nucleic acid sequence of 7-15 nucleotide residues; each R^{AM} is independently an attachment moiety capable of attaching covalently or noncovalently to a matrix-forming agent; L is a bond or a linker moiety; and m is an integer from 1 to 4.

In some embodiments, the DNA-1 comprises at least one thymine (T). In some embodiments, the forming in (c) comprises forming a covalent bond between a 3′-OH of the DNA-1 and the 5′-phosphate group of the RNA under the catalysis of a ligase. In some embodiments, a covalent bond between a 3′-OH of the DNA-1 and the 5′-phosphate group of the RNA is formed under the catalysis of a ligase. In some embodiments, the ligase is an RNA ligase. In some embodiments, the ligase is T4 RNA Ligase 1.

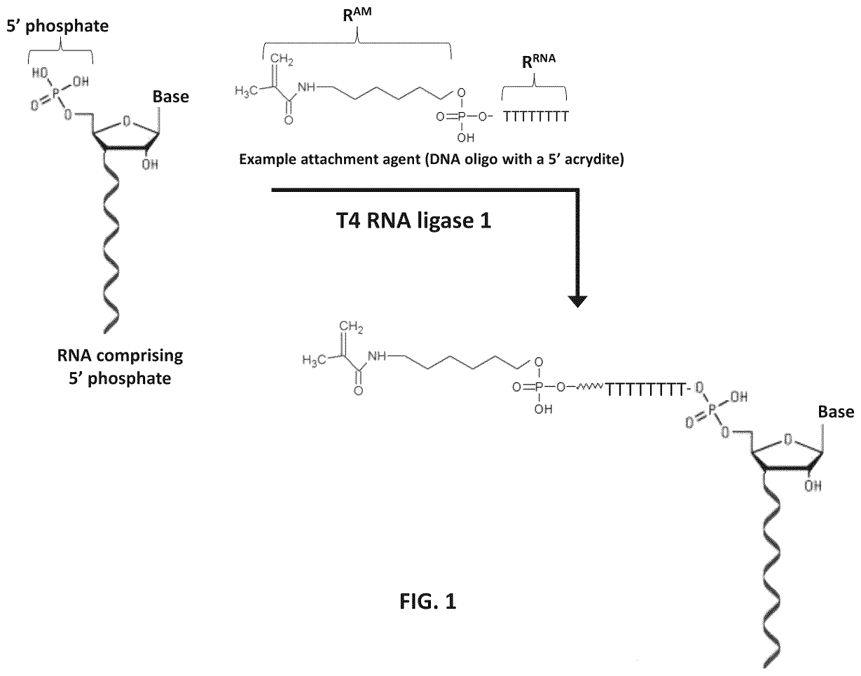

An embodiment of a reaction between a reactive moiety R^{RNA} with an RNA with a 5′-phosphate through an enzymatic process is illustrated in FIG. 1. In some embodiments (e.g., as illustrated in FIG. 1), the attachment moiety R^{AM} is an acrydite. In some embodiments (e.g., as illustrated in FIG. 1), the reactive moiety R^{RNA} is a nucleic acid, such as a DNA. In the embodiment shown in FIG. 1, the DNA is a sequence of thymine residues. However, the nucleic acid oligonucleotide (e.g., DNA oligonucleotide) can comprise any sequence. In some embodiments, the nucleic acid oligonucleotide does not comprise a sequence of thymines. In some embodiments, the nucleic acid oligonucleotide comprises or is a random sequence. As shown in FIG. 1, in some embodiments the reactive moiety R^{RNA} (e.g., a nucleic acid comprising a 3′ hydroxyl group) reacts with an RNA comprising a 5′-phosphate under the catalysis of an RNA ligase, such as T4 RNA Ligase 1. In the example illustrated, R^{RNA} is a nucleic acid comprising 8 thymine (T).

In some embodiments, at least one reactive moiety is capable of reacting with at least one 5′-phosphate group or 5′-phosphate group modified with a leaving group of the RNA via a non-enzymatic reaction. In some embodiments, at least one reactive moiety is capable of reacting with a 5′-phosphate group modified with a leaving group of the RNA via a non-enzymatic reaction, such a substitution reaction.

In some embodiments, at least one reactive moiety comprises or is a nucleophilic group capable of reacting with 5′-phosphate group modified with a leaving group of the ribonucleic acid. In some embodiments, at least one reactive moiety comprises or is an amine moiety, an amide moiety, an alcohol moiety, a thiol moiety, a cyano moiety, an ylide moiety, a hydrazide, a hydroxylamine, a hydrazine, a thiosemicarbazone, a hydrazine carboxylate, or an arylhydrazide, or any combination thereof. In some embodiments, at least one reactive moiety is or comprises an amine moiety, an amide moiety, an alcohol moiety, a thiol moiety, a cyano moiety, or an ylide moiety.

In some embodiments, at least one reactive moiety is or comprises an amine moiety (e.g., —NHR or —NH₂), an alcohol moiety, or a thiol moiety that is capable of reacting with a 5′-phosphate group modified with a leaving group. In some embodiments, the reactive moiety of the attachment agent is or comprises an amine moiety (e.g., —NHR or —NH₂). In some embodiments, the reaction of an amine moiety with a 5′-phosphate group modified with a leaving group of the ribonucleic acid forms a P—NH or —P—NR bond. In some embodiments, the reactive moiety of the attachment agent is or comprises an alcohol moiety (e.g., —OH). In some embodiments, the reaction of an alcohol moiety with a 5′-phosphate group modified with a leaving group of the ribonucleic acid forms a P—O bond. In some embodiments, the reactive moiety of the attachment agent is or comprises a thiol moiety (e.g., —SH). In some embodiments, the reaction of a thiol moiety with the 5′-phosphate group modified with a leaving group of the ribonucleic acid forms a P—S bond.

In some embodiments, the method comprises anchoring both a 5′ end and a 3′ end of an RNA to the biological sample or to a matrix embedding the biological sample. In some instances, the method comprises anchoring a 5′ end of an RNA to the biological sample or to a matrix embedding the biological sample according to any of the embodiments described herein, and further comprises anchoring the 3′ end of the RNA to the matrix. In some embodiments, anchoring the 3′ end of the RNA to the matrix comprises contacting the biological sample with a probe that hybridizes to the 3′ end of the RNA (e.g., a probe comprising a plurality of thymine bases that hybridizes to a polyA tail of the RNA). In some embodiments, anchoring the 3′ end of the RNA comprises contacting the biological sample comprising the RNA with a formylation reagent, wherein the RNA comprises a 2′,3′-vicinal diol and the formylation reagent converts the 2′,3′-vicinal diol moiety into a 2′3′-dialdehyde moiety; and contacting the biological sample with a 3′-end attachment agent comprising at least one aldehyde-reactive group capable of reacting with at least one aldehyde of the 2′,3′-dialdehyde moiety of the ribonucleic acid to form a covalent bond and an attachment moiety capable of attaching covalently or non-covalently to an exogenous or endogenous molecule in the biological sample (e.g., to a matrix).

In some embodiments, the 3′-end attachment agent comprises an aldehyde-reactive group. In some embodiments, the aldehyde-reactive group comprises or is a nucleophilic group capable of reacting with at least one aldehyde of the 2′,3′-dialdehyde moiety of the ribonucleic acid. In some embodiments, the reactive group comprises or is an amine moiety, an amide moiety, an alcohol moiety, a thiol moiety, a cyano moiety, an ylide moiety, a hydrazide, a hydroxylamine, a hydrazine, a thiosemicarbazone, a hydrazine carboxylate, or an arylhydrazide, or any combination thereof. In some embodiments, the aldehyde-reactive group or first reactive group of the attachment agent is or comprises an amine moiety, an amide moiety, an alcohol moiety, a thiol moiety, a cyano moiety, or an ylide moiety. In some embodiments, the aldehyde-reactive group of the 3′-end attachment agent is or comprises an amine moiety (e.g., —NHR or —NR2). In some embodiments, the reaction of an amine moiety with an aldehyde moiety of the ribonucleic acid forms an imine or an enamine. In some embodiments, the method comprises reduction of the imine (e.g., with NaBH₄, optionally with 0.2M NaBH₄).

In some instances, the biological sample is enzymatically treated to polish (e.g., end repair) the 3′ ends into diols and add 5′ phosphates to the RNAs. In some instances, the biological sample is contacted with a T4 PNK and to polish (e.g., end repair) the 3′ ends into diols and add 5′ phosphates to the RNAs. In some embodiments, after T4 PNK polishing, the method comprises incubating the biological sample with a ligase and an attachment agent according to any of the embodiments of Formula (I-a) described herein (e.g., a tethering oligonucleotide comprising a 5′ acrydite). In some embodiments, the biological sample is contacted with between about 10 nM and about 100 nM of the tethering oligonucleotide, optionally wherein the biological sample is contacted with about 20 nM of tethering oligonucleotide. In some embodiments, the method comprises incubating the biological sample with the tethering oligonucleotide, ligase, and a PEG reagent (e.g., PEG8000) at a temperature of about 25° C. for about 1 hour to about 4 hours (e.g., at least about 1, 1.5, or 2 hours). In some embodiments, the method further comprises tethering the polished 3′ end of the RNA by RNA oxidation (e.g., with NaIO₄, optionally with 20 mM NaIO₄), aldehyde coupling (e.g., with 2AEM, methacrylamide, Et₃N, and aniline), and imine reduction (e.g., with NaBH₄, optionally with 0.2M NaBH₄).

In some embodiments as illustrated in FIG. 2 and FIG. 3B, the 5′-phosphate group modified with a leaving group are contacted with an attachment agent illustrated in the FIG. 2 and FIG. 3B, comprising an amine moiety (e.g., —NH₂), an alcohol moiety, or a thiol moiety, to form a link between the attachment agent and the RNA.

In some embodiments, contacting the biological sample comprising an RNA and attachment agent further comprises contacting the biological sample with one or more reagents or under suitable conditions to facilitate the formation of a covalent bond between the 5′-phosphate group modified with a leaving group of the ribonucleic acid and the reactive moiety of the attachment agent. For example, in some embodiments, the methods provided herein comprise contacting the attachment agent and the biological sample with aniline and/or triethylamine to facilitate formation of a covalent bond between the 5′-phosphate group modified with a leaving group of the ribonucleic acid and the reactive moiety of the attachment agent. In some embodiments, the methods comprise contacting the attachment agent and the biological sample with triethylamine. In some embodiments, the methods comprise contacting the attachment agent and the biological sample with triethylamine. In some embodiments, the methods do not comprise contacting the attachment agent and the biological sample with triethylamine to facilitate formation of a covalent bond between the 5′-phosphate group modified with a leaving group of the ribonucleic acid and the reactive moiety of the attachment agent. In some embodiments, the formation of a covalent bond between the 5′-phosphate group modified with a leaving group of the ribonucleic acid and optionally the reactive moiety of the attachment agent is further facilitated by an inorganic salt. In some embodiments, the inorganic salt is MgCl₂. In some embodiments, the formation of a covalent bond between the 5′-phosphate group modified with a leaving group of the ribonucleic acid and the reactive moiety of the attachment agent is facilitated by shifting the equilibrium to the right, optionally by removing the products after the substitution reaction. For example, in some embodiments when the leaving group is

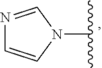 [SMILES: *n1ccnc1]

the reaction is facilitated by selectively removing 1H-imidazole from the reaction system.

In some embodiments, the leaving group is any leaving group discussed in Section II-A: Ribonucleic Acid(s) provided herein.

ii. Attachment Moiety

In some embodiments, the attachment moiety is any functional group that interacts with a matrix-forming agent and, in some embodiments, the attachment moiety comprises or is a group capable of reacting with, covalently binding, or non-covalently binding to a complementary reactive group on the matrix-forming agent.

In some embodiments, at least one attachment moiety is capable of attaching covalently to a matrix-forming agent. In some embodiments, at least one attachment moiety is or comprises an alkenyl, alkynyl, allyl or vinyl moiety, ally ester moiety, an acrylamide moiety, an amide moiety, an alcohol moiety, a polyol moiety, a furan moiety, a maleimide moiety, a norbornene moiety, a thiol moiety, a sulfide moiety, a phenol moiety, a urethane moiety, a cyano moiety, an amino moiety, an isocyanate moiety, an isothiocyanate moiety, an ether moiety, a dextran moiety, or an alginate moiety.

In some embodiments, at least one attachment moiety comprises or is an electrophilic group that is capable of interacting with a reactive nucleophilic group present on the matrix-forming agent to provide a covalent bond between the attachment moiety and the matrix-forming agent. In some embodiments, the nucleophilic groups on the matrix-forming agent having that capability include but are not limited to, sulfhydryl, hydroxyl and amino functional groups. In some embodiments, at least one attachment moiety comprises or is a maleimide, haloacetamide, or NHS ester.

In some embodiments, at least one attachment moiety comprises or is a nucleophilic group that is capable of interacting with a reactive electrophilic group present on the matrix-forming agent to provide a covalent bond between the attachment moiety and the matrix-forming agent. In some embodiments, at least one attachment moiety comprises or is a thiol, phenol, amino, hydrazide, hydroxylamine, hydrazine, thiosemicarbazone, hydrazine carboxylate, or arylhydrazide. In some embodiments, each attachment moiety is independently or independently comprises a phenol moiety, an alkyne moiety, a norbornene moiety, a sulfide moiety, a furan moiety, a maleimide moiety, or an allyl ester moiety.

In some embodiments, at least one attachment moiety comprises or is a click functional group. Suitable click functional groups include functional groups compatible with a nucleophilic addition reaction, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, and Cu(I)-catalyzed azide-alkyne cycloaddition (CuAAC) reaction. In some embodiments, the attachment moiety(ies) or second reactive group comprise or is any functional group involved in click reactions. In some embodiments, such click reactions involve (i) azido and cyclooctynyl; (ii) azido and alkynyl; (iii) tetrazine and dienophile; (iv) thiol and alkynyl; (v) cyano and amino thiol; (vi) nitrone and cyclooctynyl; or (vii) cyclooctynyl and nitrone. It should be recognized that in instances in which the attachment moiety comprises or is a click functional group, the matrix-forming agent to which it is capable of forming a covalent bond comprises the complementary click functional group to that of the attachment moiety. For example, in some embodiments, the attachment moiety comprises or is an azide moiety and the matrix-forming agent comprises a complementary alkyne moiety, or vice versa.

In some embodiments, at least one attachment moiety comprises or is a group capable of reacting with a matrix-forming agent. As detailed herein, examples of matrix-forming agents include but are not limited to acrylamide, bis-acrylamide, polyacrylamide and derivatives thereof, poly(ethylene glycol) and derivatives thereof. (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof. In some embodiments, the matrix-forming agent is or comprises acrylamide, bis-acrylamide, polyacrylamide or derivatives thereof. In some embodiments, the matrix-forming agent is or comprises acrylamide. In some embodiments, the matrix-forming agent is or comprises methylcellulose. In some embodiments, the matrix-forming agent is or comprises agarose. In some embodiments, the matrix-forming agent is or comprises collagen. In some embodiments, at least one attachment moiety comprises or is an alkenyl, allyl or vinyl moiety, an amide moiety, an alcohol moiety, a polyol moiety, a furan moiety, a maleimide moiety, a norbornene moiety, a thiol moiety, a phenol moiety, a urethane moiety, a cyano moiety, an isocyanate moiety, an isothiocyanate moiety, an ether moiety, a dextran moiety, or an alginate moiety. In some embodiments, at least one attachment moiety comprises or is an alkenyl, allyl or vinyl moiety (e.g., —C═C— or HC═C— or HC═C—CH₂—), such as in N-(2-aminoethyl)methacrylamide, 2-aminoethyl methacrylate, 2-aminoethyl (E)-but-2-enoate, 2-aminoethyl methacrylate or methylacrylamide, or norbomene. Such alkenyl, allyl or vinyl moieties may be suitable for reaction with matrix-forming agents.

In some embodiments, at least one attachment moiety comprises or is an acrylate moiety, methacrylate moiety, acrylamide moiety, methacrylamide moiety, biotinyl moiety, dextrin moiety, a click moiety, a thiol moiety, norbornenyl moiety, furanyl moiety, alkyl ester moiety, or maleimidyl moiety. In certain embodiments, at least one attachment moiety comprises or is a biotinyl moiety, dextrin moiety, a click moiety, a thiol moiety, norbornenyl moiety, furanyl moiety, alkyl ester moiety, or maleimidyl moiety.

In some embodiments, the attachment agent is of Formula (I-b):

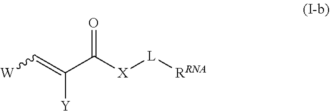

wherein R^{RNA} is any of the reactive moiety provided herein;
wherein L is a bond or any of the linker moiety provided herein;
wherein W is independently H or C₁₋₆ alkyl;
wherein Y is H or C₁₋₆ alkyl; and
wherein X is NH, N(C₁₋₆ alkyl), or O.

In some embodiments, the attachment agent is of Formula (I-c):

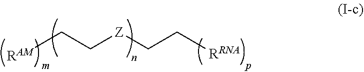

wherein n is an integer from 0 to 50;
wherein Z is CH₂ or 0;
m is an integer from 1 to 4;
p is an integer from 1 to 4;
wherein R^{RNA} is any of the reactive moiety provided herein; and wherein R^{AM} is phenol, azide, alkyne, norbornene, sulfide, furan, maleimide, or allyl ester.

In some embodiments, the formation of a covalent or non-covalent bond between the attachment moiety and the matrix-forming agent is mediated by an external reagent or stimulus. For example, in some embodiments, the formation of a covalent or non-covalent bond between the attachment moiety and the matrix-forming agent is initiated or induced by an enzyme, a catalyst, chemical reagents (e.g., acid, base, reducing agent, oxidant, etc.), heat, and/or light. In some embodiments, a covalent bond is formed between the attachment moiety and the matrix-forming agent. In some embodiments, contacting the biological sample and attachment agent further comprises contacting the biological sample with one or more reagents or under suitable conditions to facilitate the formation of a covalent bond between at least one attachment moiety of the attachment agent and the matrix-forming agent. For example, in some embodiments wherein the attachment moiety comprises an alkene or a click functional group, the method optionally further comprises adding reagents to activate the alkene or click functional group, such as a radical initiator or a copper catalyst, respectively. In other embodiments wherein at least one attachment moiety comprises an alkenyl, allyl or vinyl moiety, the method optionally further comprises exposing the biological sample and attachment agent to (ultraviolet) light or heat to facilitate formation of a covalent bond. In some embodiments wherein at least one attachment moiety comprises or is a norbornene moiety, furan moiety, maleimide moiety, or other alkenyl, allyl or vinyl moiety, the method optionally further comprises exposing the sample to light or heat. In yet other embodiments, the method may further comprise adding an enzyme to facilitate formation of a covalent bond. For example, in some embodiments wherein at least one attachment moiety comprises or is a phenol moiety, the method optionally further comprises adding horseradish peroxidase (HRP).

In some embodiments, the attachment moiety is capable of attaching non-covalently to a matrix-forming agent. In some embodiments, the attachment moiety is capable of attaching non-covalently to an exogenous molecule in the biological sample. In some embodiments, the attachment moiety is capable of attaching non-covalently to an endogenous molecule in the biological sample. In some embodiments, the attachment moiety comprises or is a group capable of binding to a matrix-forming agent via non-covalent interaction, such as but not limited to hydrogen bonding, van der Waals interaction, and/or pi-stacking.

In some embodiments, the attachment agent is biotinylated. In some embodiments, the attachment moiety is a biotin moiety or a derivative thereof.

iii. Linker L

In some embodiments, such as some embodiments of Formula (I), (I-a), or (I-b), L is a bond. In some embodiments of Formula (I), L is a linker moiety. In some embodiment, L comprises or is an unbranched or branched C₁-C₁₅₀ alkylene, which is interrupted by 1 to 50 independently selected O, NH, N, S, C₆-C₁₂ arylene, or 5- to 12-membered heteroarylene. In some embodiments, L comprises or is an unbranched and uninterrupted C₁-C₁₅₀ alkylene. In some embodiments, L comprises or is a branched and uninterrupted C₁-C₁₅₀ alkylene. In some embodiments, L comprises or is an unbranched C₁-C₁₅₀ alkylene interrupted by 1 to 50 NH, O, or S. In some embodiments, L comprises or is

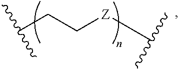

Z is CH₂, O, S; or NH; and n is an integer between 0 and 50. In some embodiments, L is

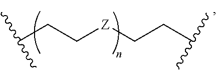

Z is CH₂, O, S; or NH; and n is an integer between 0 and 50 (e.g., between 0 and 30, between 0 and 20, between 0 and 10, or any of 4, 5, 6, 7, 8, 9). In some embodiments, L comprises or is

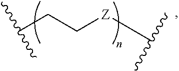

Z is CH₂, O, S; or NH; and n is an integer between 1 and 10. In some embodiment, L comprises or is

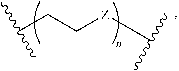

Z is CH₂, O, S; or NH; and n is 6. In some embodiments, L comprises or is an unbranched C₁-C₁₅₀ alkylene interrupted by 1 to 50 oxygen. In some embodiments, L comprises a polyethylene glycol portion or is a polyethylene glycol moiety. In some embodiments, L comprises or is

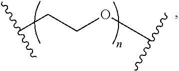

and n is an integer between 0 and 50. In some embodiments, L comprises or is

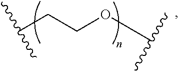

and n is an integer between 1 and 10. In some embodiment, L is

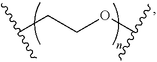

and n is 6. In some embodiment, L is

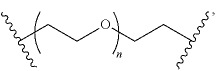

and n is an integer between 0 and 50 (e.g., between 0 and 30, between 0 and 20, between 0 and 10, or any of 4, 5, 6, 7, 8, 9). In some embodiments, L comprises an oligoethylene glycol. In some embodiments, L is an oligoethylene glycol moiety. In some embodiments, L comprises or is a branched C₁-C₁₅₀ alkylene interrupted by 1 to 50 oxygen. In some embodiments, L comprises or is an unbranched C₁-C₁₅₀ alkylene interrupted by 1 to 50 sulfurs. In some embodiments, L comprises or is

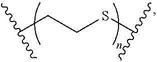

and n is an integer between 0 and 50. In some embodiment, L is

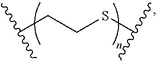

and n is 6. In some embodiment, L is

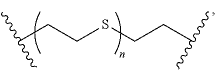

and n is an integer between 0 and 50 (e.g., between 0 and 30, between 0 and 20, between 0 and 10, or any of 4, 5, 6, 7, 8, 9). In some embodiments, L comprises or is a branched C₁-C₁₅₀ alkylene interrupted by 1 to 50 sulfurs. In some embodiments, L comprises or is a branched C₁-C₁₅₀ alkylene interrupted by 1 to 50 —NH—. In some embodiments, L comprises or is an unbranched C₁-C₁₅₀ alkylene interrupted by 1 to 50 —NH—. In some embodiments, L comprises or is

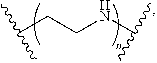

and n is an integer between 0 and 50. In some embodiment, L is

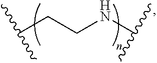

and n is 6. In some embodiment, L is

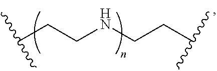

and n is an integer between 0 and 50 (e.g., between 0 and 30, between 0 and 20, between 0 and 10, or any of 4, 5, 6, 7, 8, 9). In some embodiments, L is a branched C₁-C₁₅₀ alkylene interrupted by 1 to 50 —NH—, wherein the —NH— is not at a branching point. In some embodiments, L comprises or is a branched C₁-C₁₅₀ alkylene interrupted by 1 to 50 —N—, wherein the —N— is at a branching point. In some embodiments, L comprises or is an unbranched or branched C₁-C₁₅₀ alkylene interrupted by 1 to 50 independently selected C₆-C₁₂ arylene, for example, any of phenyl or naphthalene. In some embodiments, L comprises or is an unbranched or branched C₁-C₁₅₀ alkylene interrupted by 1 to 50 independently selected 5- to 12-membered heteroarylene, for example, any of pyridine, furan, pyrrole, or thiophene.
iv. Exemplary Formulae

In some embodiments, the attachment agent as described herein (e.g., the attachment agent of Formula (I)) comprises any of 1, 2, 3, or 4 reactive groups (e.g., R^{RN}A). The reactive groups R^{RNA} of Formula (I) are each independently selected and as defined in subsection (C) (i) above. In some embodiments, the attachment agent is a bifunctional molecule comprising one reactive group. In some embodiments of Formula (I), p is any of 1, 2, 3, or 4. In some embodiments, the attachment agent (e.g., Formula (I)) comprises more than one reactive groups R^{RNA} (e.g., p is any of 2, 3, or 4), wherein the R^{RNA} groups are the same group, selected from the embodiments provided herein. In some embodiments, the attachment agent (e.g., Formula (I)) comprises more than one reactive groups R^{RNA} (e.g., p is any of 2, 3, or 4), wherein each R^{RNA} is independently selected from the embodiments provided herein, provided the more than one reactive groups are chemically compatible and have chemically compatible ribonucleic-binding mechanisms or reactions.

In some embodiments, the attachment agent (e.g., Formula (I)) comprises any of 1, 2, 3, or 4 attachment moieties (e.g., R^{A}M). The attachment moieties R^{AM} of Formula (I) are each independently selected and as defined in subsection (C)(ii) above.

In some embodiments, R^{AM} is capable of reacting with a matrix-forming agent to form a covalent bond. In some embodiments, R^{AM} is capable of reacting with a matrix-forming agent. In some embodiments, R^{AM} is In some embodiments, R^{AM} is an alkenyl, allyl or vinyl moiety, an amide moiety, an alcohol moiety, a polyol moiety, a furan moiety, a maleimide moiety, a norbomene moiety, a thiol moiety, a phenol moiety, a urethane moiety, a cyano moiety, an isocyanate moiety, an isothiocyanate moiety, an ether moiety, a dextran moiety, or an alginate moiety. In some embodiments, R^{AM} is a biotin moiety or a derivative thereof. In some embodiments, R^{AM} is an acrylate moiety, methacrylate moiety, acrylamide moiety, methacrylamide moiety, biotinyl moiety, dextrin moiety, a click moiety, a thiol moiety, norbornenyl moiety, furanyl moiety, alkyl ester moiety, or maleimidyl moiety. In certain embodiments, R^{AM} is a biotinyl moiety, dextrin moiety, a click moiety, a thiol moiety, norbornenyl moiety, furanyl moiety, alkyl ester moiety, or maleimidyl moiety.

In some embodiments of Formula (I), m is any of 1, 2, 3, or 4. In some embodiments, the attachment agent (e.g., Formula (I)) comprises more than one attachment moieties R^{AM} (e.g., m is any of 2, 3, or 4), wherein the attachment moieties are the same group, selected from the embodiments provided herein. In some embodiments, the attachment agent (e.g., Formula (I)) comprises more than one attachment moieties R^{AM} (e.g., m is any of 2, 3, or 4), wherein each R^{AM} is independently selected from the embodiments provided herein, provided the more than one reactive groups are chemically compatible and their binding mechanism or reactions to the matrix-forming agent are also chemically compatible.

In some embodiments, the attachment agent is a compound of Formula (I-d),

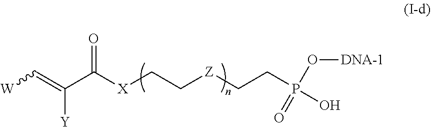

or a salt thereof, wherein
W is independently H or C₁₋₆ alkyl (e.g., CH₃);
Y is H or C₁₋₆ alkyl (e.g., CH₃);
X is NH, N(C₁₋₆ alkyl), or O;
Z is CH₂, O, or S;
n is an integer from 0 to 50 (e.g., an integer between 1 and 20 or between 1 and 10); and
DNA-1 comprises a nucleic acid sequence of 7-15 nucleotide residues.

In some embodiments, the attachment agent is a compound of Formula (I-e),

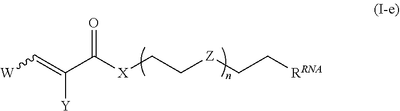

or a salt thereof, wherein
W is independently H or C₁₋₆ alkyl (e.g., CH₃);
Y is H or C₁₋₆ alkyl (e.g., CH₃);
X is NH, N(C₁₋₆ alkyl), or O;
Z is CH₂, O, or S;
n is an integer from 0 to 50 (e.g., an integer between 1 and 20 or between 1 and 10); and
R^{RNA} is NH₂, OH, or SH.

In some embodiments, the attachment agent is of Formula (I-f):

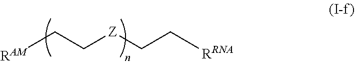

wherein n is an integer from 0 to 50 (e.g., an integer between 1 and 20 or between 1 and 10);
wherein Z is CH₂ or 0;
wherein R^{RNA} is NH₂ or OH; and
wherein R^{AM} is phenol, azide, alkyne, norbomene, sulfide, furan, maleimide, or allyl ester.

In some embodiments of any of Formulae (I-c), (I-d), (I-e), and (I-f), L is

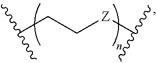

Z is O and n is 6. In some embodiments wherein L is

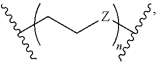

L is a hexaethylene glycol moiety.

### D. Exogenous or Endogenous Molecule(s)

In some embodiments, the methods of the present disclosure comprise immobilization of a ribonucleic acid modified with an attachment agent having an attachment moiety capable of forming a covalent or non-covalent bond to a matrix-forming agent.

In some embodiments, the attachment moiety is capable of attaching covalently to a matrix-forming agent. In some embodiments, the attachment moiety is capable of attaching covalently to a matrix-forming agent. In some embodiments, the attachment moiety is capable of attaching non-covalently to a matrix-forming agent.

It should be recognized that the attachment agent (and attachment moiety thereof) and the matrix-forming agent are selected with respect to one another, such that the matrix-forming agent has a functional group capable of ligating or bonding to the attachment moiety of the attachment agent. In some embodiments, the matrix-forming agent comprises a complementary functional group capable of bonding covalently or non-covalently to the attachment moiety of the attachment agent. For example, in some embodiments wherein the attachment agent is N-(2-aminoethyl)methacrylamide, 2-aminoethyl methacrylate, 2-aminoethyl (E)-but-2-enoate, or methacrylamide, the matrix-forming agent is an acrylamide monomer. In some embodiments wherein the attachment moiety is biotin or the attachment agent is biotinylated, the matrix-forming agent is streptavidin.

In some embodiments, the matrix-forming agent is any of the matrix-forming agent described in Section IV, capable of forming a three-dimensional polymerized matrix under suitable reaction conditions. Suitable matrix-forming agents are described herein. For example, in some embodiments, the matrix-forming agent comprises polyacrylamide, cellulose, alginate, polyamide, cross-linked agarose, cross-linked dextran or cross-linked polyethylene glycol. In some embodiments wherein the exogenous molecule is a matrix-forming agent, the method further comprises contacting the biological sample with a matrix-forming agent; and forming a three-dimensional polymerized matrix from the matrix-forming agent, thereby embedding the biological sample in the three-dimensional polymerized matrix and anchoring the ribonucleic acid to the three-dimensional polymerized matrix. In some embodiments wherein a three-dimensional polymerized matrix has been formed from the matrix-forming agent, the method optionally further comprises clearing the biological sample embedded in the three-dimensional polymerized matrix. In some embodiments, biological sample is cleared with a detergent, a lipase, and/or a protease.

As detailed herein, in some embodiments, contacting the matrix-forming agent and attachment agent further comprises contacting the biological sample with one or more reagents or under suitable conditions to facilitate the formation of a covalent bond between the second reactive group of the attachment agent and the matrix-forming agent. For example, in some embodiments wherein the attachment moiety comprises an alkenyl, the method optionally further comprises reagents to activate the alkenyl or click functional group, such as a radical initiator for polymerization.

For example, in some embodiments, the three-dimensional polymerized matrix is formed by subjecting the matrix-forming agent to polymerization. In some embodiments, the polymerization is initiated by adding a polymerization-inducing catalyst, UV light or functional cross-linkers. In some embodiments, the method further comprises staining, permeabilizing, cross-linking, expanding, and/or de-cross-linking the biological sample embedded in the three-dimensional polymerized matrix. In some embodiments, the method further comprises staining, permeabilizing, cross-linking, expanding, and/or de-cross-linking the biological sample embedded in the three-dimensional polymerized matrix after the attachment moiety has been covalently or non-covalently bonded to the matrix-forming agent.

### III. Methods for Repairing and Analyzing Fragmented RNA

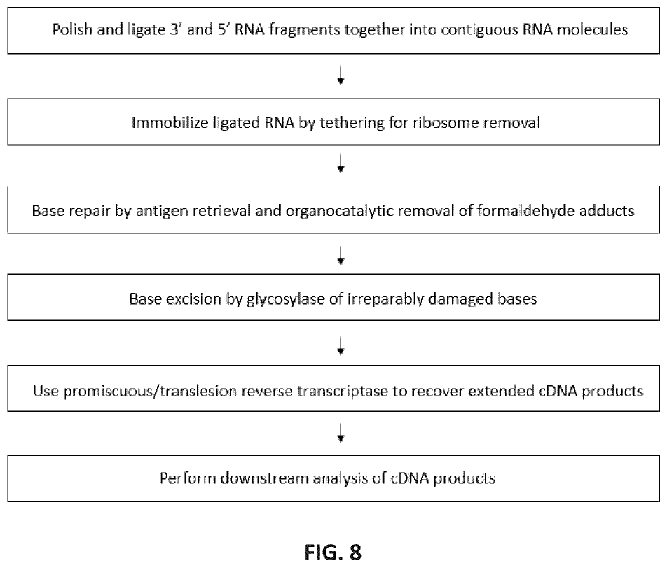

In some embodiments, provided herein are methods for repairing and analyzing fragmented RNA in a biological sample. In some embodiments, herein are provided methods for improving RNA quality from a biological sample for downstream applications. In some embodiments, herein is provided a workflow for repairing RNA molecules in a biological sample such as a formalin-fixed, paraffin-embedded biological sample. In some embodiments, the RNA molecules to be repaired have undergone transesterification breakage reactions. In some embodiments, the RNA molecules to be repaired comprise one or more nucleotide base adducts. An example of a workflow for repairing fragmented RNA is illustrated in FIG. 8. In some instances, a repaired and ligated RNA is tethered to a matrix embedding the biological sample. In some instances, after repairing and generating a ligated RNA, the biological sample is cleared. In some embodiments, the RNA fragments are repaired and ligated to generate a ligated RNA at a location in a biological sample and then released from the biological sample for downstream analysis. For example, the repair and ligation are performed at a location to allow the polished 3′ RNA fragment and a nearby polished 5′ RNA fragment (e.g., within a few nm of each other) in the biological sample to be ligated, such that the close proximity allows fragments that were originally part of the same molecule prior to being fragmented to be joined.

### A. Methods for Processing RNA Fragments

In some embodiments, herein are provided methods for polishing (e.g., end repairing) RNA fragments in a biological sample. In some embodiments, the RNA fragments are mRNA fragments. In some embodiments, the biological sample is a tissue section, optionally wherein the biological sample is a fixed tissue section. In some embodiments, the fixed tissue section is a formalin-fixed, paraffin-embedded (FFPE) tissue section. In some embodiments, the RNA fragments in the tissue section comprise one or more phosphodiester breakages, optionally wherein the one or more phosphodiester breakages are caused by one or more fixation steps. In some embodiments, the one or more phosphodiester breakages yields 3′ RNA fragments and 5′ RNA fragments in the biological sample.

In some embodiments, enzymatic repair or “polishing” of RNA fragments improves the success of subsequent attempts at RNA repair via ligation. In some embodiments, RNA fragments generated from phosphodiester breakages require chemical modification or polishing in order to allow for subsequent ligation. In some embodiments, enzymatic polishing of RNA fragments allows for subsequent ligation of polished RNA fragments, improving yields for downstream applications such as in situ, spatial array capture, single-cell based analysis, and/or sequencing of RNA transcripts.

i. Repairing 3′ Ends of RNA Fragments

In some embodiments, provided herein are methods for polishing a 3′ end of an RNA fragment in a biological sample. In some embodiments, the 3′ end of the RNA fragment results from RNA hydrolysis, wherein a phosphodiester bond in the sugar-phosphate backbone of RNA breaks and yields a fragmented 3′ end and a fragmented 5′ end, for example, as shown in FIG. 5A. In some embodiments, the fragmented 3′ end results from the breakage of a phosphodiester bond in the sugar-phosphate backbone of the RNA molecule. In some embodiments, the 3′ RNA fragment comprises a 2′3′ cyclophosphate or a 3′ phosphate. In some embodiments, the 2′3′ cyclophosphate or the 3′ phosphate specifically prevents any attempts at ligating the 3′ end of the RNA fragment with a proximal 5′ end of another RNA fragment, such as for RNA repair. In some embodiments, the 3′ end of the RNA fragment comprises a 2′3′ cyclophosphate. In some embodiments, the 2′3′ cyclophosphate inhibits attempts at ligating the 3′ end of the RNA fragment with a proximal 5′ end of another RNA fragment. In some embodiments, the 3′ end of the RNA fragment comprises a 3′ phosphate. In some embodiments, the 3′ phosphate inhibits attempts at ligating the 3′ end of the RNA fragment to a proximal 5′ end of another RNA fragment. Examples of 3′ ends of RNA fragments that inhibit end repair are shown in FIG. 5B.

In some embodiments, the 3′ end of an RNA fragment is enzymatically polished such that it can be ligated to a proximal 5′ RNA fragment comprising a 5′ phosphate. In some embodiments, the 3′ end of an RNA fragment comprising a 2′3′ cyclophosphate is enzymatically polished. In some embodiments, the 3′ end of an RNA fragment comprising a 3′ phosphate is enzymatically polished. In some embodiments, enzymatically polishing the 3′ end of the RNA fragment yields a polished 3′ end of an RNA fragment, wherein the polished 3′ end of the RNA fragment comprises a 3′ hydroxyl.

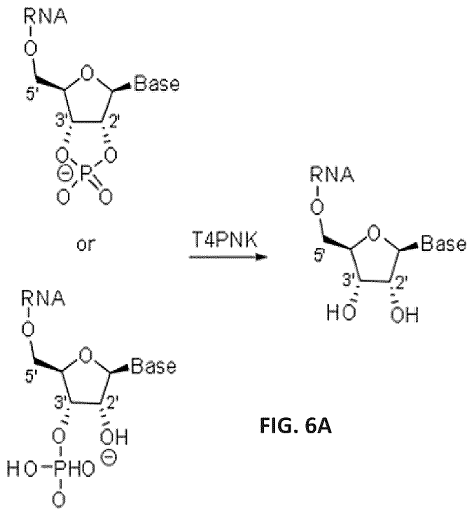

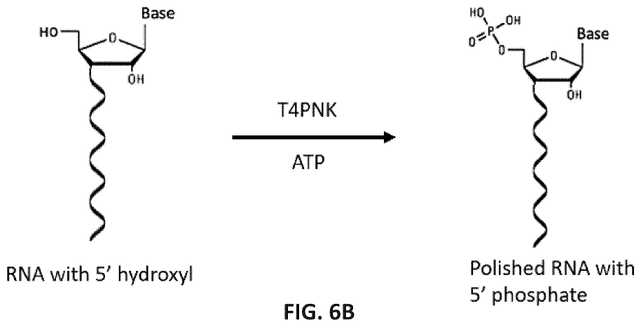

In some embodiments, a 3′ end of an mRNA fragment in the biological sample is polished with a 3′ phosphatase, for example, as shown in FIG. 6A. In some embodiments, the 3′ phosphatase polishes the 3′ end of the mRNA fragment comprising a 3′-phosphate or a 2′3′-cyclophosphate to yield a polished 3′ mRNA fragment comprising a 3′ hydroxyl. In some embodiments, the 3′ phosphatase is T4 polynucleotide kinase. In some embodiments, the T4 polynucleotide kinase polishes a 3′ end of an mRNA fragment comprising a 3′-phosphate or a 2′3′-cyclophosphate to yield a polished 3′ end of the mRNA fragment comprising a 3′ hydroxyl. In some embodiments, a T4 PNK polishes the 3′ cyclophosphate to yield a 3′ hydroxyl in the biological sample. In some embodiments, a T4 PNK polishes the 3′ phosphate to yield a 3′ hydroxyl in the biological sample. In some embodiments, the polished 3′ end of the mRNA fragment is subsequently ligated to a polished 5′ end of another mRNA fragment comprising a 5′ phosphate to yield a repaired mRNA fragment.

In some embodiments, a 3′ end of a fragment of a first, second, and/or third fragment of a cellular RNA comprises a 2′3′ cyclophosphate prior to end-repairing. In some embodiments, the 3′ end of the first, second, and/or third fragment of the cellular RNA comprises a 3′ phosphate prior to the end-repairing. In some embodiments, the end-repairing comprises a 3′ phosphatase enzymatically polishing the 3′ fragment of the first, second, and/or third fragment of a cellular RNA to yield a 2′3′ vicinol diol.

ii. Repairing 5′ Ends of RNA Fragments

As detailed herein, the methods of the present disclosure encompass methods for enzymatically polishing a 5′ end of an RNA fragment, such as a 5′ end of an mRNA fragment, in a biological sample. Common mRNA breakages, such as those produced via transesterification, result in a 5′ end of an RNA fragment comprising a hydroxyl group and a 3′ end of an RNA fragment comprising a 3′-phosphate or 2′3′-cyclophosphate, wherein the 3′ end of the fragment, in particular, inhibits any attempts at backbone repair. FIG. 5A shows an example of a transesterification reaction resulting in a 3′ fragment and a 5′ fragment.

In some embodiments, a 5′ end of an mRNA fragment in the biological sample is repaired with a 5′ kinase, for example, as shown in FIG. 6B. In some embodiments, the 5′ kinase polishes the 5′ end of the mRNA fragment comprising a 5′ hydroxyl to yield a polished 5′ end of the mRNA fragment comprising a 5′ phosphate. In some embodiments, the 5′ kinase is T4 polynucleotide kinase. In some embodiments, the T4 polynucleotide kinase polishes the 5′ mRNA fragment comprising the 5′ hydroxyl to yield a repaired 5′ end of an mRNA fragment comprising a 5′ phosphate. In some embodiments, the polished 5′ end of the mRNA fragment can subsequently be ligated to a repaired 3′ mRNA fragment comprising a 3′ hydroxyl to yield a repaired mRNA fragment.

In some embodiments, the polishing of the 3′ end of an RNA fragment and the polishing of the 5′ end of the same RNA fragment and/or another RNA fragment may occur simultaneously or in one contacting step. In some embodiments, the tissue sample is contacted with a T4 polynucleotide kinase, wherein the T4 polynucleotide kinase polishes both a 3′ RNA fragment and a 5′ RNA fragment.

In some embodiments, a 5′ fragment of a first, second, and/or third fragment of a cellular RNA comprises a 5′ hydroxyl prior to end-repairing. In some embodiments, the end-repairing comprises a 3′ phosphatase enzymatically polishing the 5′ fragment of the first, second, and/or third fragment of a cellular RNA to yield a 5′ phosphate. In some embodiments, the end-repaired 5′ fragment can then be ligated together with a repaired 3′ end fragment comprising a 2′3′ vicinal diol.

iii. Phosphatases and Kinases

In some embodiments, herein is provided a 3′ phosphatase and a 5′ kinase for repairing RNA fragments in a biological sample such as a FFPE tissue section. In some embodiments, the 3′ phosphatase polishes a 3′ RNA fragment comprising a 2′3′ cyclophosphate or a 3′ phosphate to yield a polished 3′ RNA fragment comprising a 2′3′ vicinal diol. In some embodiments, the 3′ phosphatase is T4 PNK. In some embodiments, the 5′ kinase is T4 PNK. In some embodiments, T4 PNK acts as both a 3′ phosphatase and a 5′ kinase in the biological sample. In some embodiments, the 3′ phosphatase is T7 PNK. In some embodiments, the 5′ kinase is T7 PNK. In some embodiments, T7 PNK acts as both a 3′ phosphatase and a 5′ kinase in the biological sample. In some embodiments, the biological sample is contacted with a 3′ phosphatase and a 5′ kinase in a buffer comprising magnesium ions.

B. Removing Ribosomes from Tethered RNA Fragments

In some embodiments, the biological sample is embedded in a matrix, and the method comprises partially or substantially clearing the matrix of certain species or classes of biomolecules, such as lipids and proteins, e.g., by use of detergent and/or protease reagents. In some embodiments, the repaired RNA is tethered to the matrix. In some embodiments, the repaired RNA is tethered to the matrix after further processing (e.g., repairing base adducts) as described in Section III.C. According to some aspects of the present disclosure, the sample is cleared using a detergent solution, such as Triton-X or SDS. The detergent can interact with the molecules allowing the molecules to be washed out or removed. Other non-limiting examples of detergents include Triton X-100, Triton X-114, Tween-20, Tween 80, saponin, CHAPS, and NP-40. According to some aspects of the present disclosure, the sample can be cleared using a protease reaction, such as Proteinase K. In some instances, the protease cleaves or digests proteins such that the fragments or amino acids can be removed. In some instances, the extracellular matrix is substantially cleared using one or more specific or nonspecific proteases. Other non-limiting examples of protease include trypsin, chemotrypsin, papain, thrombin, and pepsin.

In some embodiments, herein are provided methods for removing ribosomes from a biological sample following tethering or immobilization of RNA fragments in the biological sample. In some embodiments, ribosomes may act as steric blocking factors that impede access to the RNA molecule in the biological sample. In some embodiments, ribosomes are cleared from the biological sample. In some embodiments, the ribosomes are cleared in order to increase access to the RNA molecule in the biological sample. In some embodiments, the method comprises removing ribosomes from the tethered RNA by treating the biological sample with a detergent and a protease after forming a matrix embedding the biological sample. In some embodiments, the detergent comprises SDS and the protease comprises proteinase K. In some embodiments, the detergent and the protease are provided in a buffer of at least pH 8.0. In some embodiments, the biological sample is treated with the detergent and the protease at at least 45° C. for no more than 4 minutes. In some embodiments, the biological sample is treated with the detergent and the protease at about 50° C. for about 3 minutes. In some embodiments, the biological sample is treated with 1% SDS and 200 μg/mL proteinase K provided in a PBS buffer of at least pH 8.5 at about 50° C. for about 3 minutes.

### C. Repairing Base Adducts in RNA Fragments

In some embodiments, an RNA molecule in the biological sample comprises a base adduct. Base adducts are damaged nucleotide bases, such as nucleotide bases that have undergone oxidation or reduction. Remarkably, modifications of every single position in the nucleobases of purines and pyrimidines in RNA have been described. Some base modifications occur via by biochemical or cellular processes. Some base modifications occur via a chemical insult.

In some embodiments, the biological sample comprises fragmented and/or damaged RNA comprising one or more RNA base adducts. In some embodiments, a base adduct comprises a base oxidation. In some embodiments, the base adduct is an adduct of a pyrimidine base. In some embodiments, the base adduct is an adduct of a purine base. In some embodiments, a pyrimidine base is oxidized at the C5 and/or the C6 positions. In some embodiments, the pyrimidine base is oxidized at the C5 position. In some embodiments, the pyrimidine base is oxidized at the C6 position.

In some embodiments, one or more nucleotide base adducts in the RNA molecule are repaired. In some embodiments, one or more nucleotide base adducts in the RNA molecule are removed from the RNA molecule. In some embodiments, one or more nucleotide base adducts in the RNA molecule are excised from the RNA molecule to yield an apurinic/apyrimidinic site (an AP site) in place of the damaged nitrogenous base that is excised in the RNA molecule.

In some embodiments, one or more nucleotide base adducts in the RNA molecule are repaired via organocatalysis. In some embodiments, repair of one or more nucleotide base adducts in the RNA molecule is performed by an organocatalyst. In some embodiments, the organocatalyst is an anthranilate. In some embodiments, the organocatalyst is a phosphanilate. Mechanisms for repair of nucleotide base adducts with water-soluble bifunctional catalysts including anthranilates and phosphanilates are described, for example, in Karmakar S et al, Organocatalytic removal of formaldehyde adducts from RNA and DNA bases, Nature Chemistry (2015), 7:752-758, the content of which is herein incorporated by reference in its entirety.

i. Guanine Adducts

In some embodiments, an RNA molecule in the biological sample comprises a guanine adduct (e.g., a damaged or chemically modified form of guanine). In some embodiments, the presence of the guanine adduct in the RNA reduces the efficacy of one or more enzymatic repair, ligation, and/or reverse transcription processes. In some embodiments, repairing or removing the guanine adduct in the RNA molecule increases the efficacy of ligation. In some embodiments, repairing or removing the guanine adduct in the RNA molecule improves recovery of RNA from the biological sample for downstream processes.

In some embodiments, an RNA base adduct in an RNA molecule in the biological sample comprises a guanine adduct. In some embodiments, a guanine nucleotide base in the RNA molecule has been converted into a guanine adduct. In some embodiments, the presence of the guanine adduct in the RNA molecule inhibits an attempt to repair the RNA molecule. In some embodiments, the presence of the guanine adduct in the RNA molecule alters one or more binding characteristics of the RNA molecule.

In some embodiments, a guanine adduct in an RNA molecule in a biological sample comprises an oxidized or hyperoxidized form of guanine. In some embodiments, the guanine in the RNA molecule in the biological sample has been oxidized or hyperoxidized into an oxidized or hyperoxidized form of guanine. In some embodiments, the guanine adduct is an oxidized form of guanine. In some embodiments, the oxidized form of guanine comprises 8-oxo-Guanine (8-oxo-Gua, alternatively referred to as 8-hydroxyguanine or OH-Gua). In some embodiments, the 8-oxo-Gua preferentially base pairs with adenine. In some embodiments, the 8-oxo-Gua preferentially base pairing with adenine results in a guanine-adenine transversion mutation. In some embodiments, the 8-oxo-Gua base pairs with cytosine. In some embodiments, the 8-oxo-Gua base pairing with cytosine comprises two hydrogen bonds instead of three. In some embodiments, the oxidized form of guanine comprises Fapy-Guanine (Fapy-Gua). In some embodiments, the hyperoxidized form of guanine comprises spiroiminohydantoin.

In some embodiments, a guanine adduct in an RNA molecule in a biological sample comprises a reduced form of guanine. In some embodiments, the guanine base in the RNA molecule in the biological sample has been reduced into a reduced form of guanine. In some embodiments, the reduced form of guanine comprises a guanine C8-OH adduct radical or a product thereof. In some embodiments, the guanine C8-OH adduct radical is further reduced to yield 2,6-diamino-4-hydroxy-5-formamidopyrimidine. In some embodiments, the reduced form of guanine comprises 2,6-diamino-4-hydroxy-5-formamidopyramidine. In some embodiments, the guanine C8-OH adduct radical is oxidized to yield 8-hydroxyguanine. In some embodiments, the reduced form of guanine comprises 8-hydroxyguanine.

In some embodiments, the guanine adduct in the biological sample is repaired. In some embodiments, the guanine adduct in the biological sample is repaired to a guanine. In some embodiments, the guanine adduct in the biological sample is repaired via organocatalysis, optionally wherein the organocatalyst is an anthranilate and/or a phosphanilate. In some embodiments, the guanine adduct in the biological sample is excised from the nucleic acid molecule. In some embodiments, the guanine adduct in the biological sample is excised, yielding an AP site. In some embodiments, repair or excision of the guanine adduct results in improved ligation and/or improved reverse transcription of the nucleic acid molecule in the biological sample.

ii. Adenine Adducts

In some embodiments, an RNA molecule in the biological sample comprises an adenine adduct (e.g., a damaged or chemically modified form of adenine). In some embodiments, the presence of the adenine adduct in the RNA reduces the efficacy of one or more enzymatic repair, ligation, and/or reverse transcription processes. In some embodiments, repairing or removing the adenine adduct in the RNA molecule increases the efficacy of ligation. In some embodiments, repairing or removing the adenine adduct in the RNA molecule improves recovery of RNA from the biological sample for downstream processes.

In some embodiments, an RNA base adduct of the fragmented and/or damaged RNA comprises an adenine adduct. In some embodiments, an adenine base within the fragmented and/or damaged RNA has been converted into an adenine adduct. In some embodiments, the presence of the adenine adduct in the fragmented and/or damaged RNA inhibits attempts to repair the fragmented and/or damaged RNA.

In some embodiments, the adenine adduct is an oxidized or hyperoxidized form of adenine. In some embodiments, the adenine adduct is an 8-oxo-adenine. In some embodiments, the 8-oxo-adenine preferentially base pairs with guanine. In some embodiments, the 8-oxo-adenine base pairs with G>T>C. In some embodiments, the presence of the 8-oxo-adenine induces an intrastrand RNA crosslink. In some embodiments, the presence of the 8-oxo-adenine disrupts double stranded configurations as short as 25 base pairs. In some embodiments, the adenine adduct is a Fapy-adenine. In some embodiments, the adenine adduct is a 2-oxo-adenine. In some embodiments, the adenine adduct is a deaminated adenine. In some embodiments, the adenine adduct is an inosine. In some embodiments, the inosine is a promiscuous base. In some embodiments, the inosine is capable of forming hydrogen bonds with all types of nucleotide bases. In some embodiments, the inosine is capable of forming hydrogen bonds with guanine, adenine, cytosine, and uracil bases, and/or with modified nucleotide bases present in the biological sample.

In some embodiments, the adenine adduct in the biological sample is repaired. In some embodiments, the adenine adduct in the biological sample is repaired to an adenine. In some embodiments, the adenine adduct in the biological sample is repaired via organocatalysis, optionally wherein the organocatalyst is an anthranilate and/or a phosphanilate. In some embodiments, the adenine adduct in the biological sample is excised from the nucleic acid molecule. In some embodiments, the adenine adduct in the biological sample is excised, yielding an AP site. In some embodiments, repair or excision of the adenine adduct results in improved ligation and/or improved reverse transcription of the nucleic acid molecule.

iii. Cytosine Adducts

In some embodiments, an RNA molecule in the biological sample comprises a cytosine adduct (e.g., a damaged or chemically modified form of cytosine). In some embodiments, the presence of the cytosine adduct in the RNA reduces the efficacy of one or more enzymatic repair, ligation, and/or reverse transcription processes. In some embodiments, repairing or removing the cytosine adduct in the RNA molecule increases the efficacy of ligation. In some embodiments, repairing or removing the cytosine adduct in the RNA molecule improves recovery of RNA from the biological sample for downstream processes.

In some embodiments, an RNA base adduct of the fragmented and/or damaged RNA is a cytosine adduct. Cytosine, a pyrimidine base, is universally susceptible to oxidation on the C5 and C6 positions. In some embodiments, the cytosine is oxidized at the C5 position. In some embodiments, the cytosine is oxidized at the C6 position. In some embodiments, the cytosine is oxidized at both the C5 position and the C6 position. In some embodiments, a cytosine base within the fragmented and/or damaged RNA has been converted into a cytosine adduct. In some embodiments, the presence of the cytosine adduct in the fragmented and/or damaged RNA inhibits attempts to repair the fragmented and/or damaged RNA.

In some embodiments, the cytosine adduct is a 5-OH-cytosine. In some embodiments, the 5-OH-cytosine leads to an abasic site in the backbone. In some embodiments, the cytosine adduct is an Imid-Cyt adduct. In some embodiments, the Imid-Cyt is unable to hydrogen bond with other nucleotide bases. In some embodiments, the cytosine adduct is a 5-CaCyt. In some embodiments, the cytosine adduct is a 5-Fo-Cyt. In some embodiments, the cytosine adduct is a 5-HmCyt.

In some embodiments, the cytosine adduct in the biological sample is repaired. In some embodiments, the cytosine adduct in the biological sample is repaired to a cytosine. In some embodiments, the cytosine adduct in the biological sample is repaired via organocatalysis, optionally wherein the organocatalyst is an anthranilate and/or a phosphanilate. In some embodiments, the cytosine adduct in the biological sample is excised from the nucleic acid molecule. In some embodiments, the cytosine adduct in the biological sample is excised, yielding an AP site. In some embodiments, repair or excision of the adenine adduct results in improved ligation and/or improved reverse transcription of the nucleic acid molecule.

iv. Uracil Adducts

In some embodiments, an RNA molecule in the biological sample comprises a uracil adduct (e.g., a damaged or chemically modified form of uracil). In some embodiments, the presence of the uracil adduct in the RNA reduces the efficacy of one or more enzymatic repair, ligation, and/or reverse transcription processes. In some embodiments, repairing or removing the uracil adduct in the RNA molecule increases the efficacy of ligation. In some embodiments, repairing or removing the uracil adduct in the RNA molecule improves recovery of RNA from the biological sample for downstream processes.

In some embodiments, an RNA base adduct in the biological sample is a uracil adduct. Uracil, a pyramidine base, is universally susceptible to oxidation on the C5 and C6 positions. In some embodiments, the uracil is oxidized at the C5 position. In some embodiments, the uracil is oxidized at the C6 position. In some embodiments, the uracil is oxidized at both the C5 position and the C6 position. In some embodiments, a uracil base within the fragmented and/or damaged RNA has been converted into a uracil adduct. In some embodiments, the presence of the uracil adduct in the fragmented and/or damaged RNA inhibits or impairs attempts to repair the fragmented and/or damaged RNA.

In some embodiments, the uracil adduct is a pseudouradine. In some embodiments, the uracil adduct is a UraGly adduct. In some embodiments, the UraGly adduct results in an abasic site in the backbone. In some embodiments, the uracil adduct is a 5-methyluradine. In some embodiments, the uracil adduct is a 5-hydroxymethyluradine.

In some embodiments, fixation of a biological sample (for example, fixation of a formalin-fixed, paraffin-embedded tissue section) can result in the addition of hemiaminal and/or aminal products to adenine, cytosine, or guanine. In some embodiments,

In some embodiments, the uracil adduct in the biological sample is repaired. In some embodiments, the uracil adduct in the biological sample is repaired to a uracil. In some embodiments, the uracil adduct in the biological sample is repaired via organocatalysis, optionally wherein the organocatalyst is an anthranilate and/or a phosphanilate. In some embodiments, the uracil adduct is excised from the nucleic acid molecule. In some embodiments, the uracil adduct in the biological sample is excised, yielding an AP site. In some embodiments, repair or excision of the uracil adduct results in improved ligation and/or improved reverse transcription of the nucleic acid molecule.

v. Excision of Irreparably Damaged Bases

In some embodiments, one or more irreparably damaged bases in an RNA fragment are excised. In some embodiments, the excision of one or more irreparably damaged RNA bases is performed via glycosylase. In some embodiments, a glycosylase enzyme excises one or more irreparably damaged RNA bases from an RNA molecule in the biological sample. In some embodiments, the irreparably damaged RNA base is a guanine adduct. In some embodiments, the guanine adduct excised from the RNA molecule is an 8-oxo-guanine, a Fapy-guanine, a spiroiminodihydantoin, a guanine C8-OH adduct radical, a 2,6-diamino-4-hydroxy-5-formamidopyrimidine, or a 8-hydroxyguanine. In some embodiments, the irreparably damaged RNA base is a cytosine adduct. In some embodiments, the cytosine adduct excised from the RNA molecule is a 5-OH-cytosine, an imid-cytosine, a 5-Ca-Cyt, a 5-FoCyt, or a 5-HmCyt. In some embodiments, the irreparably damaged RNA base is an adenine adduct. In some embodiments, the adenine adduct excised from the RNA molecule is an 8-oxo-adenine, a Fapy-adenine, a 2-oxo-adenine, or an inosine. In some embodiments, the irreparably damaged RNA base is a uracil adduct. In some embodiments, the uracil adduct excised from the RNA molecule is a pseudoeuridine, a ura-gly, a 5-methyluradine, or a 5-hydroxymethyluradine.

In some embodiments, a glycosylase enzyme removes a damaged nitrogenous base from the RNA molecule while leaving intact the sugar-phosphate backbone of the RNA molecule. In some embodiments, the glycosylase leaves an apurinic/apyrimidinic site (an AP site) in the RNA molecule in place of the damaged nitrogenous base that is excised. In some embodiments, excising one or more damaged bases within an RNA fragment results in improved ligation of the RNA fragment.

In some embodiments, the glycosylase enzyme is an AlkA glycosylase enzyme, optionally an AlkA derived from E. coli. In some embodiments, the glycosylase enzyme is an MBD4. In some embodiments, the glycosylase enzyme is NEIL1. In some embodiments, the glycosylase enzyme is an enzyme selected from a group consisting of: APE1, Endo III, Tma Endo III, Endo IV, Tth Endo IV, Endo V, Endo VIII, Fpg, hAAG, T4 PDG, UDG, Afu UDG, Antarctic Thermolabile UDG, Warmstart® Afu Uracil-DNA Glycosylase (UDG), hSMUG1, and Thermostable OGG.

### D. Ligation of Polished RNA Fragments

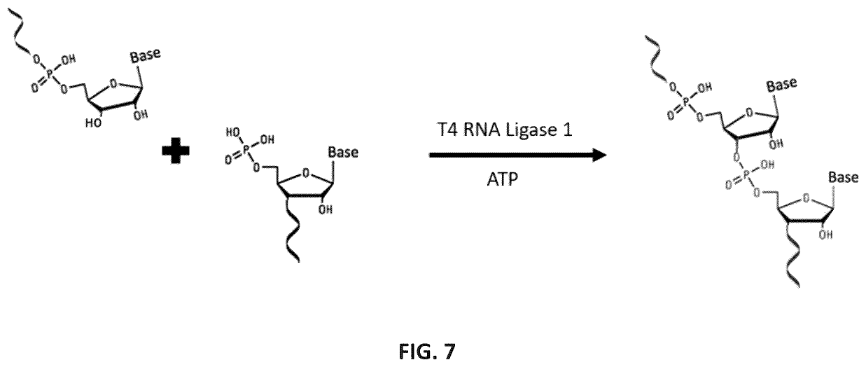

In some embodiments, the polished 3′ RNA fragment and the polished 5′ RNA fragment are ligated together in the biological sample to form a repaired RNA. Ligation of polished RNA fragments wherein the 3′ fragment comprises a 2′3′ vicinal diol is shown, for example, in FIG. 7.

In some embodiments, the polished 3′ RNA fragment and the polished 5′ RNA fragment are located at the same location in the biological sample. In some embodiments, the polished 3′ RNA fragment and the polished 5′ RNA fragment are within 10 nm, 9 nm, 8 nm, 7 nm, 6 nm, 5 nm, 4 nm, 3 nm, 2 nm, 1 nm, or less of the same location within the biological sample. In some embodiments, physical proximity of the polished 3′ RNA fragment and the polished 5′ RNA fragment within the biological sample is a requirement for successful ligation. In some embodiments, the requirement for physical proximity of the polished 3′ RNA fragment and the polished 5′ RNA fragment in the biological sample in order for ligation to occur ensures that only RNA fragments from the same RNA molecule are ligated together in the biological sample. In some embodiments, the requirement for physical proximity of the polished 3′ RNA fragment and the polished 5′ RNA fragment in the biological sample in order for ligation to occur ensures that RNA fragments originating from distinct RNA molecules are not erroneously ligated together.

i. Ligases

In some embodiments, the polished 3′ RNA fragment and the polished 5′ RNA fragment are ligated together with an RNA ligase. In some embodiments, the RNA ligase is a T4 RNA ligase. In some embodiments, the T4 RNA ligase is a T4 RNA ligase 1. In some embodiments, the RNA ligase is any suitable RNA ligase, for example, as described in Section IV-C. In some embodiments, the ligase is capable of ligating together a polished 3′ RNA fragment and a polished 5′ RNA fragment when the polished 3′ RNA fragment and the polished 5′ RNA fragment are at or near the same location in the biological sample, and wherein the polished 3′ RNA fragment comprises a 2′3′ vicinal diol. In some embodiments, the polished 3′ RNA fragment and the polished 5′ RNA fragment are within 1, 2, 3, 4, 5, 6, 7, 8, 9, 10 or fewer nm of each other in the biological sample.

In some embodiments, the ligase ligates a polished 3′ RNA fragment and a polished 5′ RNA fragment at a location in the biological sample to yield a repaired RNA molecule. In some embodiments, the ligase does not ligate a polished 3′ RNA fragment and a polished 5′ RNA fragment if the polished 3′ RNA fragment and the polished 5′ RNA fragment are not proximally located in the biological sample. In some embodiments, the polished 3′ RNA fragment and the polished 5′ RNA fragment are within 1, 2, 3, 4, 5, 6, 7, 8, 9, or 10 nm of each other in the biological sample and can be ligated together. In some embodiments, the ligase ligates a polished 3′ RNA fragment and a polished 5′ RNA fragment wherein the polished 3′ RNA fragment and the polished 5′ RNA fragment are located proximally to one another in the biological tissue.

In some embodiments, the RNA ligase is not capable of ligating together an unpolished 3′ RNA fragment and an unpolished 5′ RNA fragment, wherein the unpolished 3′ RNA fragment does not comprise a 2′3′ vicinal diol. In some embodiments, enzymatic polishing of the 3′ RNA fragment and/or the 5′ RNA fragment prior to ligation is required for successful ligation of the 3′ RNA fragment and the 5′ RNA fragment. In some embodiments, enzymatic polishing of the 3′ RNA fragment to yield a 2′3′ vicinal diol is required in order for the RNA ligase to ligate the 3′ RNA fragment and the 5′ RNA fragment. In some embodiments, the RNA ligase ligates the polished 3′ RNA fragment and the polished 5′ RNA fragment to generate a repaired RNA molecule in the biological sample.

### E. Downstream Analysis of Repaired RNA

In some embodiments, the repaired RNA molecule is subjected to further processing and/or analysis. In some embodiments, the repaired RNA molecule is reverse transcribed into a cDNA product for downstream processing applications. In some embodiments, downstream processing applications include but are not limited to in situ detection, spatial array-based capture and processing, detection by sequencing, and/or single-cell based detection of RNA and/or cDNA products generated from RNA in the biological sample. In some embodiments, the tethered or immobilized RNA is detected as described in Section IV.C.

i. Reverse Transcription

In some embodiments, the method further comprises performing reverse transcription on the repaired RNA to generate a cDNA product. In some embodiments, the method further comprises performing reverse transcription on the ligated RNA to generate a cDNA product from the ligated RNA. In some embodiments, the reverse transcription is performed using a promiscuous reverse transcriptase enzyme. In some embodiments, the reverse transcription is performed using a trans-lesion reverse transcriptase enzyme. In some embodiments, the reverse transcription is performed using a reverse transcriptase enzyme with low fidelity. In some embodiments, the reverse transcription is performed using a trans-lesion reverse transcriptase enzyme with low fidelity. In some embodiments, the reverse transcriptase is HIV-1 RT. In some embodiments, the reverse transcriptase is Murine Leukemia Virus RT.

In some embodiments, use of a low-fidelity and/or translesion reverse transcriptase enzyme increases the length of the reverse transcribed molecules across abasic sites and otherwise chemically modified bases in the RNA. In some embodiments, use of a low-fidelity and/or translesion reverse transcriptase enzyme results in longer cDNA products compared to the use of a reverse transcriptase enzyme that is not low-fidelity. In some embodiments, use of a low-fidelity and/or translesion reverse transcriptase enzyme results in longer cDNA products compared to the use of a reverse transcriptase enzyme that is not a translesion reverse transcriptase enzyme. In some embodiments, the use of a low-fidelity and/or translesion reverse transcriptase enzyme results in greater processivity compared to the use of a reverse transcriptase enzyme that is not low-fidelity. In some embodiments, the use of a low-fidelity and/or translesion reverse transcriptase enzyme results in greater processivity compared to the use of a reverse transcriptase enzyme that is not a translesion reverse transcriptase enzyme.

In some embodiments, the reverse transcription occurs in the presence of a nucleotide mixture comprising modified nucleotide bases. In some embodiments, the nucleotide mixture comprising modified nucleotide bases comprises 5-methylcytosine and/or pseudouridine nucleotide analogs to the RT reaction. In some embodiments, the nucleotide mixture comprising modified nucleotide bases comprises other chemically stabilized nucleotide derivatives. In some embodiments, the addition of modified bases such as 5-methylcytosine and pseudouridine nucleotide analogs may enhance abasic site crossings during reverse transcription by providing stability and structural support. In some embodiments, one or more modified nucleotides are incorporated into the RNA molecule prior to ligation and/or reverse transcription. In some embodiments, the one or more modified nucleotides increase the stability of the RNA molecule during reverse transcription.

In some embodiments, the reverse transcription yields a cDNA product that can be further analyzed. In some embodiments, the cDNA product is analyzed in situ in the biological sample. In some embodiments, the cDNA product is analyzed on a substrate. In some embodiments, the cDNA product is analyzed on a spatial array. In some embodiments, the cDNA product is further processed and/or analyzed in a cell partition.

ii. In Situ Analysis of Repaired mRNA

In some embodiments, the repaired RNA and/or the cDNA generated from the repaired RNA are analyzed in situ in the biological sample. In some embodiments, the repaired RNA and/or cDNA are tethered to the biological sample or a matrix embedding the biological sample (e.g., by crosslinking of the repaired RNA and/or cDNA or of a probe that binds to the repaired RNA and/or cDNA). In some embodiments, the method comprises contacting the ligated RNA or the cDNA with a circularizable probe (e.g., a padlock probe). In some embodiments, the method comprises circularizing the circularizable probe. In some embodiments, the method comprises circularizing the padlock probe. In some embodiments, the method comprises performing rolling circle amplification with the circularized probe as a template. In some embodiments, the method comprises performing rolling circle amplification with the circularized padlock probe as a template. In some embodiments, the method comprises using a probe to detect a sequence of or associated with the repaired RNA and/or the cDNA in the rolling circle amplification product in the biological sample. In some embodiments, the rolling circle amplification product is tethered in the biological sample.

In some embodiments, the method further comprises contacting the biological sample with a probe or probe set that binds directly or indirectly to the ligated RNA or to the cDNA generated from the ligated RNA. In some embodiments, the method comprises detecting the bound probe or probe set or a product thereof. In some embodiments, the probe or probe set is a circular or circularizable probe or probe set. In some embodiments, the method comprises circularizing the circularizable probe or probe set using the ligated RNA or the cDNA generated from the ligated RNA as a template. In some embodiments, the method comprises generating an RCA product using the circular or circularizable probe as a template. In some embodiments, the method comprises imaging the biological sample to detect the probe or probe set or the RCA product. In some embodiments, imaging the biological sample comprises detecting a signal associated with the probe or probe set or the RCA product. In some embodiments, the signal associated with the probe or probe set or the RCA product is detected at a location in the biological sample. In some embodiments, the signal is from a fluorescently labeled probe that directly or indirectly binds to the probe or probe set or the RCA product. In some embodiments, the probe or probe set comprises a barcode sequence that identifies the cellular RNA, optionally wherein the method comprises detecting the barcode sequence or a complement thereof in the probe or probe set or in a product of the probe or probe set. In some embodiments, the ligated RNA or to the cDNA generated from the ligated RNA is detected as described in Section IV.C.

iii. Single-Cell Capture

In some embodiments, a workflow provided herein is incorporated into a single-cell RNA sequencing workflow in order to increase RNA recovery from a biological sample. In some embodiments, methods provided herein are used to improve RNA capture from low-quality biological samples. For example, the repaired RNA as described in Section III is in a biological sample that is then dissociated into single cells and a single cell barcoding reaction is performed.

In some embodiments, the biological sample comprises a cell and the cell is partitioned. In some instances, the cell comprises the repaired RNA. In some embodiments, the partition is a microwell or a droplet. In some embodiments, the droplet is an emulsion droplet. In some embodiments, the partition comprises a support that comprises a plurality of barcode oligonucleotides comprising a partition barcode sequence and a capture sequence. In some embodiments, the barcode oligonucleotides are releasably attached to the support. In some embodiments, the support is a bead. In some embodiments, the bead is a gel bead. In some embodiments, the capture sequence binds to the ligated RNA or a product thereof. In some embodiments, using a barcode oligonucleotide of the plurality of barcode oligonucleotides to generate a barcoded cDNA product of the ligated RNA comprising a sequence of the partition barcode sequence or complement thereof. In some embodiments, generating the barcoded cDNA product comprises performing reverse transcription of the captured ligated RNA using a promiscuous or trans-lesion reverse transcriptase enzyme. In some embodiments, the reverse transcriptase is HIV-1 RT. In some embodiments, the reverse transcriptase is Murine Leukemia Virus RT. In some embodiments, the reverse transcription is performed in the presence of a nucleotide mixture comprising 5-methylcytosine and/or pseudouridine nucleotide analogs to the RT reaction. In some embodiments, a barcoded product comprising a sequence of the repaired RNA or associated with the repaired RNA is generated. In some cases, the barcoded product comprises a sequence corresponding to the repaired RNA or a complement thereof and a sequence of the partition barcode sequence or complement thereof.

In some embodiments, the method further comprises releasing the barcoded cDNA product or a complement thereof from the partition. In some embodiments, the method further comprises pooling the barcoded cDNA product or complement thereof from the partition with contents of other partitions of a plurality of partitions. In some embodiments, the method further comprises amplifying the barcoded cDNA product or complement thereof. In some embodiments, the method further comprises determining a sequence of the barcoded cDNA product or complement thereof.

In some embodiments, a single cell barcoding reaction is performed and comprises a templated ligation reaction. For example, a templated ligation process comprises contacting a nucleic acid molecule (e.g., a ligated RNA generated from RNA fragments that are repaired and ligated as described in Section III) with a probe molecule, such as a DNA probe, RNA probe, or a probe comprising both DNA and RNA. In some instances, the probe molecule interacts with one or more other nucleic acid molecules, for example, those comprising a barcode sequence, to generate a probe-barcode complex. In some instances, an extension reaction is performed on at least a portion of the probe-barcode complex to generate a nucleic acid product that comprises the barcode sequence and is associated with a sequence of the ligated and repaired RNA. In some cases, barcoding of the nucleic acid molecule is performed without reverse transcription on the ligated RNA. In some instances, the methods herein comprise ligation-mediated reactions.

In some embodiments, repairing RNA in the biological sample according to any of the methods provided herein improves RNA capture for single-cell RNA sequencing applications. In some embodiments, the methods provided herein are advantageous for rescuing RNA quality for single-cell RNA sequencing applications.

iv. Spatial Array Capture

In some embodiments, repairing RNA in the biological sample is performed according to any of the methods provided herein and the repaired RNA (or a product or derivative thereof) is transferred to a substrate (e.g., an array) for further processing. In some embodiments, the method comprises capturing the ligated RNA on an array. In some embodiments, the method comprises sequencing all or a portion of the captured ligated RNA or a complement thereof, optionally wherein the captured ligated RNA or complement thereof is amplified. In some embodiments, the method comprises performing reverse transcription of the captured ligated RNA to generate a cDNA product of the captured ligated RNA on the array. In some embodiments, the reverse transcription is performed using a promiscuous or trans-lesion reverse transcriptase enzyme. In some embodiments, the reverse transcriptase is HIV-1 RT. In some embodiments, the reverse transcriptase is Murine Leukemia Virus RT. In some embodiments, the reverse transcription is performed in the presence of a nucleotide mixture comprising 5-methylcytosine and/or pseudouridine nucleotide analogs to the RT reaction.

In some embodiments, the method comprises sequencing all or a portion of the cDNA or a complement thereof, optionally wherein the cDNA or complement thereof is amplified. In some embodiments, the method comprises capturing the cDNA on an array. In some embodiments, the method comprises sequencing all or a portion of the captured cDNA or a complement thereof, optionally wherein the captured cDNA or complement thereof is amplified. In some embodiments, the method comprises generating an amplification product of the captured ligated RNA or complement thereof comprising a spatial barcode sequence or complement thereof that identifies the location of the captured ligated RNA on the array. In some embodiments, the method comprises generating an amplification product of the captured cDNA or complement thereof comprising a spatial barcode sequence or complement thereof that identifies the location of the captured cDNA on the array.

In some embodiments, a plurality of repaired RNAs or derivatives thereof are detected on an array or by nucleic acid sequencing. In some embodiments, the method comprises generating a spatially barcoded oligonucleotide comprising (i) a sequence of the repaired RNA or product thereof or complement thereof and (ii) a sequence of the spatial barcode sequence or complement thereof and releasing the spatially barcoded oligonucleotide from the substrate (e.g., array) for sequencing. In some embodiments, the substrate is an array. For example, the repaired RNA (or a derivative thereof) can be captured by an oligonucleotide (e.g., a capture probe immobilized, directly or indirectly, on a substrate) comprising a capture sequence complementary to a sequence of a probe set used to detect the repaired RNA or a derivative thereof on an array, optionally amplified, and sequenced, thus determining the location and optionally the abundance of the analyte in the biological sample.

In some cases, spatial analysis is performed by detecting multiple oligonucleotides (e.g., probes or probe sets) that hybridize to a target nucleic acid (e.g., a repaired RNA). In some instances, for example, spatial analysis is performed by hybridization of two oligonucleotides (e.g., a first probe and a second probe) to adjacent sequences on an analyte (e.g., the repaired RNA). In some instances, the oligonucleotides are DNA molecules. In some instances, the probe or probe set includes a capture domain (e.g., a poly(A) sequence, a non-homopolymeric sequence). In some embodiments, after hybridization to the analyte, a ligase (e.g., SplintR ligase) ligates the two oligonucleotides (e.g., of a probe set) together, creating a ligation product (e.g., a ligated probe set that forms a single linear molecule). In some instances, the two or more molecules of the probe set hybridize to sequences that are not directly adjacent to one another. For example, in some embodiments, hybridization of the two probes of a probe set creates a gap between the hybridized nucleic acid molecules. In some instances, a polymerase (e.g., a DNA polymerase) can extend one of the nucleic acid molecules prior to ligation. After ligation, the ligation product is released from the analyte (e.g., the repaired RNA). In some instances, the ligation product is released using an endonuclease (e.g., RNAse H). The released ligation product (or a derivative thereof) can then be captured by an oligonucleotide (e.g., a capture probe immobilized, directly or indirectly, on a substrate) comprising a capture sequence complementary to a sequence of the ligated probe(s) on an array, optionally amplified, and sequenced, thus determining the location and optionally the abundance of the analyte in the biological sample. In some embodiments, the ligated probe(s) comprise a complementary capture sequence. In some instances, a ligated probe comprises an overhang region (e.g., a region that does not hybridize to the target nucleic acid) comprising the complementary capture sequence. In some embodiments, the capture sequence comprises a polyA sequence. In some embodiments, the oligonucleotide (e.g., capture probe) comprising the capture sequence comprises a spatial barcode sequence. In some embodiments, the method comprises generating a spatially barcoded oligonucleotide comprising (i) a sequence of the ligated probe or product thereof or complement thereof and (ii) a sequence of the spatial barcode sequence or complement thereof. In some embodiments, the sequence of the ligated probe or product thereof or complement thereof corresponds to the repaired RNA.

Additional variants of spatial analysis methods, including in some embodiments, an imaging step, are described in Section (II)(a) of WO 2020/176788 and/or U.S. Patent Application Publication No. 2020/0277663, each of which is incorporated herein in its entirety. Analysis of captured analytes such as ligated probes described herein (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663, each of which is incorporated herein in its entirety. Some quality control measures are described in Section (II)(h) of WO 2020/176788 and/or U.S. Patent Application Publication No. 2020/0277663, each of which is incorporated herein in its entirety.

In some embodiments, repairing RNA in the biological sample according to any of the methods provided herein improves RNA capture for spatial array capture and downstream RNA detection. In some embodiments, the methods provided herein are advantageous for rescuing RNA quality for spatial array RNA (or a product or derivative thereof) capture and detection.

### IV. Samples, Analytes, and Detection

### A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, needle aspirate, or fine needle aspirate. In some embodiments, the biological sample is or comprises a cell pellet or a section of a cell pellet. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein is any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate is coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Preparation

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample is prepared using formalin-fixation and paraffin-embedding (FFPE), which are established methods. In some embodiments, cell suspensions and other non-tissue samples are prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein.

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, are added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Embedding

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) are embedded in a 3D matrix. In some embodiments, a 3D matrix comprises a network of natural molecules and/or synthetic molecules that are chemically and/or enzymatically linked, e.g., by crosslinking. In some embodiments, a 3D matrix comprises a synthetic polymer. In some embodiments, a 3D matrix comprises a hydrogel.

In some aspects, a biological sample is embedded in any of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof are modified to contain functional groups that can be used as an anchoring site to attach the polynucleotide probes and/or amplification product to a polymer matrix. In some embodiments, a modified probe comprising oligo dT is used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel includes hydrogel subunits, such as, but not limited to, acrylamide, bis-acrylamide, polyacrylamide and derivatives thereof, poly(ethylene glycol) and derivatives thereof. (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

In some embodiments, the hydrogel forms the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry is used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221):543-548, 2015 and U.S. Pat. No. 10,059,990, all of which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, biological samples can be stained using a wide variety of stains and staining techniques. In some embodiments, for example, a sample is stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample is segmented using one or more images taken of the stained sample.

In some embodiments, the stain is performed using a lipophilic dye. In some examples, the staining is performed with a lipophilic carbocyanine or aminostyryl dye, or analogs thereof. (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample is stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample is stained using Romanowsky stain, including Wright's stain, Jenner's stain, Can-Grunwald stain, Leishman stain, and Giemsa stain.

In some embodiments, biological samples are destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### B. Analytes

The methods disclosed herein can be used to detect and analyze a wide variety of different analytes (e.g., including RNA as described in Sections II-III and in combination with other analytes). In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g., an affinity binding partner) can be developed. Such a specific binding partner may be a nucleic acid probe (for a nucleic acid analyte).

Analytes of particular interest may include nucleic acid molecules, such as RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g., including but not limited to protein-RNA complexes, which may or may not be covalently bound to one another, and which may be the same or different. Thus, in addition to cells or microorganisms, such a complex analyte may also be a protein complex or protein interaction. Such a complex or interaction may thus be a homo- or hetero-multimer. Aggregates of molecules, e.g., proteins may also be target analytes, for example aggregates of the same protein or different proteins. The analyte may also be a complex between proteins or peptides and nucleic acid molecules such as RNA, e.g., interactions between proteins and nucleic acids, e.g., regulatory factors, such as transcription factors, and RNA.

### (i) Endogenous Analytes

In some embodiments, an analyte herein is endogenous to a biological sample and includes nucleic acid analytes and non-nucleic acid analytes. Methods and compositions disclosed herein can be used to analyze nucleic acid analytes (e.g., by immobilizing or tethering any fragmented ribonucleic acids to an endogenous molecule in the biological sample or an exogenous molecule delivered to the biological sample as described in Section II and using a nucleic acid probe or probe set that directly or indirectly hybridizes to the immobilized or tethered nucleic acid analyte).

Examples of nucleic acid analytes include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). In some embodiments described herein, an analyte is a fragmented RNA.

In certain embodiments, an analyte is extracted from a live cell. Processing conditions can be adjusted to ensure that a biological sample remains live during analysis, and analytes are extracted from (or released from) live cells of the sample. Live cell-derived analytes can be obtained only once from the sample or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence is endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

### (ii) Labelling Agents

In some embodiments, provided herein are methods and compositions for analyzing endogenous analytes (e.g., RNA) in a sample and other analytes using one or more labelling agents. In some aspects, the methods for immobilizing or tethering fragmented ribonucleic acids to an endogenous molecule in the biological sample or an exogenous molecule delivered to the biological sample as described in Section II is compatible with protein analysis (e.g., using a labelling agent). In some embodiments, an analyte labelling agent includes an agent that interacts with an analyte (e.g., an endogenous analyte in a sample). In some embodiments, the labelling agents comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., a single-stranded probe sequence), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds is also identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In some embodiments, an analyte binding moiety includes any molecule or moiety capable of binding to an analyte (e.g., a biological analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of various embodiments of labelling agents, reporter oligonucleotides, and methods of use, see, e.g., U.S. Pat. No. 10,550,429; U.S. Pat. Pub. 20190177800; and U.S. Pat. Pub. 20190367969, all of which are herein incorporated by reference in their entireties.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. In some embodiments, the oligonucleotide attached to a labelling agent comprises a sequence that serves as a primer and can be used as a reporter (e.g., a barcode). In some embodiments, the oligonucleotide attached to a labelling agent comprises both a reporter sequence (e.g., a barcode) and a sequence serving as a primer. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, the content of which is herein incorporated by reference in its entirety. Likewise, protein and peptide biotinylation techniques have been developed and are readily available. See, e.g., U.S. Pat. No. 6,265,552, the content of which is herein incorporated by reference in its entirety. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the labelling agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide or primer may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

### C. Detection and Analysis

In some aspects, the provided methods involve analyzing the tethered or immobilized RNA as described in Section II and optionally other analytes, e.g., by detecting or determining, one or more sequences present in probes or probe sets or products thereof. (e.g., rolling circle amplification products thereof). In some aspects, the provided methods involve analyzing the repaired RNA as described in Section III. In some embodiments, the detecting is performed at one or more locations in a biological sample. In some embodiments, the locations are the locations of tethered or immobilized RNA transcripts in the biological sample. In some embodiments, the locations are the locations at which probes or probe sets hybridize to the RNA transcripts in the biological sample and are optionally ligated and amplified by rolling circle amplification.

In some embodiments, the method provided herein comprises contacting the biological sample with a probe or probe set that binds directly or indirectly to the ribonucleic acid (e.g., a tethered and/or repaired fragmented RNA as described in Section II and III). In some embodiments, the probe or probe set is a detectable probe. In some embodiments, the probe or probe set comprises a barcode sequence. In some embodiments, the probe or probe set comprises an intermediate probe and a detection oligonucleotide. In some embodiments, the detecting comprises a plurality of repeated cycles of hybridization and removal of probes (e.g., detectably labeled probes, or intermediate probes that bind to detectably labeled probes) to the primary probe or probe set hybridized to the target nucleic acid, or to a rolling circle amplification product generated from the probe or probe set hybridized to the target nucleic acid.

In some embodiments, the method comprises contacting the biological sample with a circular or circularizable probe, wherein the circular or circularizable probe binds the fragmented ribonucleic acid (e.g., a tethered and/or repaired fragmented RNA as described in Section II and III) and generates a rolling circle amplification (RCA) product.

In some embodiments, the method comprises imaging the biological sample to detect the RCA product. In some embodiments, imaging comprises detecting a signal associated the probe, optionally with a fluorescently labeled probe that directly or indirectly binds to the rolling circle amplification product.

### (a) Hybridization, Ligation, and Amplification

In some embodiments, the method comprises contacting the biological sample with a probe or probe set that hybridizes directly or indirectly to the ribonucleic acid (e.g., a tethered and/or repaired fragmented RNA as described in Section II and III). For purposes of hybridization, two nucleic acid sequences are “substantially complementary” if at least 60% (e.g., at least 70%, at least 80%, or at least 90%) of their individual bases are complementary to one another. Various probes and probe sets can be hybridized to an endogenous analyte (e.g., ribonucleic acid) and/or a labelling agent.

In some embodiments, the method comprises generating a ligation product with a probe or probe set that hybridizes directly or indirectly to the ribonucleic acid (e.g., a tethered and/or repaired fragmented RNA as described in Section II and III).

In some embodiments, provided herein is a probe or probe set capable of DNA-templated ligation, such as from a cDNA molecule. See, e.g., U.S. Pat. No. 8,551,710, the content of which is herein incorporated by reference in its entirety. In some embodiments, provided herein is a probe or probe set capable of RNA-templated ligation. See, e.g., U.S. Pat. Pub. 2020/0224244, the content of which is herein incorporated by reference in its entirety. In some embodiments, the probe set is a SNAIL probe set. See, e.g., U.S. Pat. Pub. 20190055594, the content of which is herein incorporated by reference in its entirety. In some embodiments, provided herein is a multiplexed proximity ligation assay. See, e.g., U.S. Pat. Pub. 20140194311 the content of which is herein incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, the content of which is herein incorporated by reference in its entirety. In some embodiments, a circular probe is indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243, the content of which is herein incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. Without being bound by theory, “direct ligation” may comprise the ends of the polynucleotides hybridizing immediately adjacently to one another to form a substrate for a ligase enzyme resulting in their ligation to each other (intramolecular ligation). Alternatively, “indirect” can include the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe (e.g., padlock probe), or target nucleic acid. The gap may be a gap of 1 to 60 nucleotides or a gap of 1 to 40 nucleotides or a gap of 3 to 40 nucleotides. In specific embodiments, the gap is a gap of about 1, 2, 3, 4, 5, 6, 7, 8, 9 or 10 or more nucleotides of any integer (or range of integers) of nucleotides in between the indicated values. In some embodiments, the gap between said terminal regions is filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, the method comprises detecting a product that includes a molecule or a complex generated in a series of reactions, e.g., hybridization, ligation, extension, replication, transcription/reverse transcription, and/or amplification (e.g., rolling circle amplification), in any suitable combination.

In some embodiments, the detected product is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof. In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a priming strand (e.g., a primer) is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:l095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such a phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is a phi29 DNA polymerase.

In some embodiments, the tethering methods described herein are compatible with priming an extension reaction using a 3′ end of the RNA (e.g., target-primed RCA). In some embodiments, amplification of a circular probe or circularizable probe or probe set is primed by the target RNA (e.g., immobilized RNA as described in Section II). In some embodiments, the target RNA is cleaved by an enzyme (e.g., RNase H). In some embodiments, the target RNA is cleaved at a position downstream of the target sequences bound to the circular probe or circularizable probe or probe set. In some aspects, the methods disclosed herein allow targeting of RNase H activity to a particular region in a target RNA that is adjacent to or overlapping with a target sequence for a probe or probe set. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe or probe set by about 1 to about 20 nucleotides or by about 8 to about 10 nucleotides. The cleaved target RNA itself can then be used to prime RCA of the circular probe generated from a circularizable probe or probe set (e.g., target-primed RCA). In some cases, a plurality of nucleic acid oligonucleotides can be used to perform target-primed RCA for a plurality of different target RNAs.

In any of the embodiments herein, the biological sample is contacted with the RNase H (and optionally with the nucleic acid oligonucleotide) before or during formation of the circularized gap-filled first probe or probe set. In some embodiments, the biological sample is contacted with the oligonucleotide and with the RNase H simultaneously or sequentially (in either order) before contacting the sample with the probe or probe set. In any of the embodiments herein, the biological sample is contacted with the RNase H (and optionally with the nucleic acid oligonucleotide) after formation of the circularized probe or probe set. In any of the embodiments herein, the RNase H comprises an RNase H1 and/or an RNAse H2. In some embodiments, RNase inactivating agents or inhibitors are added to the sample after cleaving the target RNA.

In some aspects, the polynucleotides and/or amplification product (e.g., amplicon) are anchored to a polymer matrix. For example, the polymer matrix can be a hydrogel. In some embodiments, one or more of the polynucleotide probes are modified to contain functional groups that can be used as an anchoring site to attach the polynucleotide probes and/or amplification product to a polymer matrix. Examples of probe modifications and polymer matrix that can be employed in accordance with the provided embodiments comprise those described in, for example, WO 2014/163886, WO 2017/079406, US 2016/0024555, US 2018/0251833, US 2017/0219465, US 2023/0279484, and US 2024/0301468. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. In some embodiments, the amplification products are immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, a method disclosed herein also comprises one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, an analyte disclosed herein is detected in with a method that comprises signal amplification. Examples of signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method is used.

The detectable reactive molecules may comprise tyramide, such as used in tyramide signal amplification (TSA) or multiplexed catalyzed reporter deposition (CARD)-FISH. In some embodiments, the detectable reactive molecule is releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Examples of detectable reactive reagents and methods are described in U.S. Pat. No. 6,828,109, US 2019/0376956, US 2019/0376956, US 2021/0222234, US 2022/0026433, US 2022/0128565, WO 2020/102094, WO 2020/163397, and WO 2021/067475, all of which are incorporated herein by reference in their entireties.

In some embodiments, hybridization chain reaction (HCR) is used for in situ signal amplification and detection. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target reporter oligonucleotide. HCR is described in detail in Dirks and Pierce, 2004, PNAS, 101(43), 15275-15278 and in U.S. Pat. No. 7,632,641 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”) and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

### (b) Analysis

A target sequence for a probe or probe set disclosed herein may be comprised in any analyte disclose herein, including an endogenous analyte (e.g., a probe or probe set that hybridizes directly or indirectly to the ribonucleic acid), a labelling agent, or a product or derivative of an endogenous analyte and/or a labelling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the two or more sub-barcodes are overlapping sequences. In some embodiments, the one or more barcode(s) also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, the method comprises contacting the biological sample with a probe set that binds directly or indirectly to the ribonucleic acid. In some embodiments, the probe set comprises a plurality of primary probes comprising sequences complementary to a plurality of target sequences in the ribonucleic acid (e.g., a target ribonucleic acid). In some embodiments, the plurality of primary probes hybridize to partially overlapping target sequences in the target ribonucleic acid. In some embodiments, the plurality of primary probes hybridize to non-overlapping target sequences in the target ribonucleic acid. In some embodiments, the plurality of primary probes are designed to hybridize to 1, 2, 3, 4, 5, 6, 7, 8, 9, 10, 11, 12, 13, 14, 15 or more, 20 or more, 32 or more, 40 or more, or 50 or more target sequences tiling a target ribonucleic acid (e.g., an mRNA or fragment thereof). In some embodiments, the plurality of primary probes comprise different sequences to hybridize to the plurality of target sequences. In some cases, the plurality of primary probes are associated with the same signal (e.g., comprise the same detectable label or the same sequence for hybridization of a detectably labeled probe in an overhang region of the primary probe). In some embodiments, the method comprises fluorescent in situ hybridization to the tethered RNA.

In any of the preceding embodiments, the tethered RNA and/or a barcode associated with the tethered RNA (e.g., primary and/or secondary barcode sequences) can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing-by-binding (SBB), sequencing-by-avidity (SBA), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labelled probes (e.g., detection oligos).

In some embodiments, analyzing, e.g., detecting or determining, one or more sequences present in the biological sample is performed using a base-by-base sequencing method, e.g., sequencing-by-synthesis (SBS), sequencing-by-avidity (SBA) or sequencing-by-binding (SBB). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing is performed by sequencing-by-synthesis (SBS). In some embodiments, a sequencing primer is complementary to sequences at or near the one or more barcode(s). In such embodiments, sequencing-by-synthesis comprises reverse transcription and/or amplification in order to generate a template sequence from which a primer sequence can bind. Example SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, sequencing is performed by sequential fluorescence hybridization (e.g., sequencing by hybridization). Sequential fluorescence hybridization can involve sequential hybridization of detection probes comprising an oligonucleotide and a detectable label.

In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read is required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences rather than endogenous sequences, which is an efficient read-out in terms of information per cycle of sequencing. Because the barcode sequences are pre-determined, they can also be designed to feature error detection and correction mechanisms, see, e.g., U.S. Pat. Pub. 20190055594 and US 2021/0164039, all of which are herein incorporated by reference in their entireties.

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof. (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes can be fluorophore sequences assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In any of the embodiments herein, the detecting comprises contacting the biological sample with one or more detectably labeled probes that directly or indirectly hybridize to the barcode sequences or complements thereof. (e.g., in amplification products generated using the probes or probe sets), and dehybridizing the one or more detectably labeled probes. In any of the embodiments herein, the contacting and dehybridizing can be repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In any of the embodiments herein, the detecting comprises contacting the biological sample with one or more first detectably labeled probes that directly hybridize to the plurality of probes or probe sets. In some instances, the detecting comprises contacting the biological sample with one or more first detectably labeled probes that indirectly hybridize to the plurality of probes or probe sets. In any of the embodiments herein, the detecting comprises contacting the biological sample with one or more first detectably labeled probes that directly or indirectly hybridize to the plurality of probes or probe sets.

In any of the embodiments herein, the detecting comprises contacting the biological sample with one or more intermediate probes that directly or indirectly hybridize to the barcode sequences or complements thereof. (e.g., of the plurality of probes or probe sets or rolling circle amplification product generated using the plurality of probes or probe sets), wherein the one or more intermediate probes are detectable using one or more detectably labeled probes. In any of the embodiments herein, the detecting can further comprise dehybridizing the one or more intermediate probes and/or the one or more detectably labeled probes from the barcode sequences or complements thereof. (e.g., of the plurality of probes or probe sets or rolling circle amplification product generated using the plurality of probes or probe sets). In any of the embodiments herein, the contacting and dehybridizing can be repeated with the one or more intermediate probes, the one or more detectably labeled probes, one or more other intermediate probes, and/or one or more other detectably labeled probes.

In some embodiments, sequence detection is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, nucleic acid hybridization is used for detecting the analytes. These methods utilize labeled nucleic acid probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, a probe or probe set is a probe comprising a 3′ or 5′ overhang upon hybridization to the target nucleic acid (e.g., an L-shaped intermediate probe). In some embodiments, the overhang comprises one or more barcode sequences corresponding to the target nucleic acid (e.g., the target RNA transcript). In some embodiments, a plurality of probes are designed to hybridize to the target nucleic acid (e.g., at least 20, 30, or 40 probes can hybridize to the target nucleic acid). In some embodiments, the probe or probe set is a probe comprising a 3′ overhang and a 5′ overhang upon hybridization to the target nucleic acid (a U-shaped probe). In some embodiments, the 3′ overhang and the 5′ overhang each independently comprises one or more detectable labels and/or barcode sequences. In some embodiments, the 3′ and/or 5′ overhang comprises one or more detectable labels and/or barcode sequences.

In some embodiments, analysis comprises using a codebook comprising signal code sequence that are sequences of color codes, arranged in the order of the corresponding signal color detected in sequential cycles of probe hybridization and imaging. In some aspects, the provided methods which immobilize and tether RNA in the biological sample can be advantageous when using detection methods that comprise a plurality of repeated cycles of hybridization and removal of probes (e.g., detectably labeled probes, or intermediate probes that bind to detectably labeled probes) to the primary probe or probe set hybridized to the RNA, or to a rolling circle amplification product generated from the probe or probe set hybridized to the RNA.

### V. Kits and Systems

In some aspects, provided herein are kits or systems for analyzing fragmented nucleic acids in a biological sample. In some embodiments, provided herein are kits or systems for analyzing a ribonucleic acid in a biological sample embedded in a three-dimensional polymerized matrix according to any of the methods described herein. In some embodiments, provided herein is a kit or system comprising (a) a polynucleotide kinase, optionally wherein the polynucleotide kinase is T4 polynucleotide kinase or T7 polynucleotide kinase; (b) an attachment agent comprising at least one reactive moiety capable of reacting with at least one 5′-phosphate group of the RNA or 5′-phosphate group of the RNA modified with a leaving group and at least one attachment moiety capable of attaching covalently or noncovalently to a matrix-forming agent, optionally wherein the kit further comprises one or more reagents for reacting the attachment agent with the RNA or matrix-forming agent; (d) a matrix-forming agent capable of attaching covalently or non-covalently to the attachment moiety, optionally wherein the matrix-forming agent is for embedding the biological sample in a three-dimensional polymerized matrix; (e) a clearing agent, optionally wherein the clearing agent is a detergent, a lipase, and/or a protease; and (f) instructions for use. In some embodiments, provided herein is a kit or system comprising a matrix-forming agent configured for forming a three-dimensional polymerized matrix from the matrix-forming agent to immobilize an RNA in the three-dimensional polymerized matrix; and an attachment agent. The attachment agent can be any of the attachment agents described herein, e.g., in Section II.C. The matrix-forming agent can by any of the matrix-forming agents described herein, e.g., in Section II.C.

In some embodiments, the kit or system further comprises one or more reagents for clearing the biological sample. In some embodiments, the kit or system comprises a detergent. In some embodiments, the kit comprises a protease. In some embodiments, the detergent comprises SDS. In some embodiments, the protease comprises proteinase K. In some embodiments, the detergent and the protease are provided in a buffer of at least pH 8.0. In some embodiments, the kit comprises instructions for treating the biological sample with the detergent and the protease at a temperature of at least 45° C. for no more than 4 minutes (e.g., after attaching the RNA to the matrix). In some embodiments, the kit comprises instructions for treating the biological sample with the detergent and the protease at about 50° C. for about 3 minutes (e.g., after attaching the RNA to the matrix). In some embodiments, the kit comprises instructions for treating the biological sample with 1% SDS and 200 μg/mL proteinase K provided in a PBS buffer of at least pH 8.5 at about 50° C. for about 3 minutes (e.g., after attaching the RNA to the matrix). In some embodiments, the kit comprises instructions for detecting the RNA after treating the biological sample with the protease and detergent.

In some embodiments, the kit or system further comprises a probe or probe set designed to hybridize to a fragmented nucleic acid. In some instances, the kit or system comprises a ligase. In some instances, the kit or system comprises a polymerase for performing RCA. In some aspects, the probe or probe set that binds directly or indirectly to the ribonucleic acid is provided in a separate kit. In some cases, further provided is a kit comprising reagents for detecting the probe or probe set. In some instances, the kit or system comprises reagents for detection of the barcode sequences using sequential hybridization of probes to barcode sequences or complements thereof. In some embodiments, the kit or system further comprises one or more detectably labeled probes. In some instances, the kit or system comprises reagents for performing sequencing (e.g., sequencing by synthesis (SBS), sequencing-by-binding (SBB), sequencing-by-avidity (SBA), or sequencing by ligation (SBL). In some embodiments, the kit or system comprises a sequencing primer complementary to a sequence comprised on the probe or probe set, or a product thereof. In some embodiments, the kit or system comprises a nucleotide pool configured for performing a nucleic acid sequencing reaction.

In some aspects, provided herein are systems for repairing and/or analyzing fragmented nucleic acids in a biological sample. In some embodiments, the system comprises a polynucleotide kinase and a ligase for ligating RNA fragments. In some instances, the system further comprises a one or more tissue clearing agents. In some instances, the system further comprises an organocatalyst and/or a glycosylase. In some instances, the system further comprises one or more reagents for performing reverse transcription of the ligated RNA to generate a cDNA product of the ligated RNA (e.g., reverse transcriptase and a nucleotide mixture). In some embodiments, herein are provided kits or systems for repairing fragmented RNA in a biological sample. In some embodiments, herein are provided kits for repairing fragmented RNA in a formalin-fixed, paraffin-embedded tissue section. In some embodiments, provided herein is a kit or system comprising (a) a 3′ phosphatase, optionally wherein the 3′ phosphatase is T4 polynucleotide kinase or T7 polynucleotide kinase, (b) a 5′ kinase, optionally wherein the 5′ kinase is T4 polynucleotide kinase or T7 polynucleotide kinase, (c) an attachment agent as described in any of the above embodiments, (d) a matrix-forming agent as described in any of the preceding embodiments, (e) a clearing agent, suitable for clearing ribosomes from the biological sample, (f) an organocatalyst, optionally wherein the organocatalyst is a phosphanilate or an anthranilate, (g) a glycosylase enzyme suitable for excising irreparably damaged nucleotide bases from the RNA molecule, (h) a ligase, optionally wherein the ligase is T4 RNA ligase, optionally wherein the T4 RNA ligase is T4 RNA ligase 1, (g) a reverse transcriptase, optionally wherein the reverse transcriptase is a low-fidelity, translesion, and/or promiscuous reverse transcriptase, optionally wherein the reverse transcriptase is HIV-1 RT or Murine Leukemia Virus RT, and (h) instructions for use. In some embodiments, the kit further comprises is a nucleotide mixture comprising 5-methylcytosine and/or pseudouridine nucleotide analogs for performing the reverse transcriptase reaction.

Provided herein are systems for processing fragmented nucleic acids in a biological sample. In some embodiments, the system comprises a polynucleotide kinase and a ligase for ligating RNA fragments. In some embodiments, the kit or system comprises reagents for performing a single cell barcoding reaction (e.g., a bead, a plurality of barcode oligonucleotides). In some instances, the kit or system comprises a reverse transcriptase. In some cases, the kit or system comprises reagents for transferring the repaired RNA (or a product or derivative thereof) to a substrate (e.g., an array) and to perform downstream processing and analysis (e.g., as described in Section III.E). In some instances, the kit or system comprises reagents for generating a spatially barcoded oligonucleotide comprising (i) a sequence of the repaired RNA or product thereof or complement thereof and (ii) a sequence of the spatial barcode sequence or complement thereof. In some instances, the kit or system comprises a substrate comprising spatially barcoded oligonucleotides. In some instances, the reagents comprise oligonucleotides (e.g., a first probe and a second probe) configured to bind to adjacent sequences on an analyte (e.g., the repaired RNA). In some instances, the reagents comprise a ligase for forming a ligation product (e.g., a ligated probe set that forms a single linear molecule). In some instances, the reagents comprise a polymerase (e.g., a DNA polymerase) for extending one of the nucleic acid molecules.

In some embodiments, the various components of the kit or system are present in separate containers. In some embodiments, certain compatible components of the kit are pre-combined into a single container. In some embodiments, the kits further contain instructions for using the components of the kit to practice the provided methods.

In some embodiments, the kits contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, provided is a kit that comprises any of the reagents described herein, e.g., wash buffer and ligation buffer. In some embodiments, provided is a kit that contain reagents for detection and/or sequencing, such as barcode detection probes or detectable labels. In some embodiments, the kits contain other components, for example nucleic acid primers, enzymes and reagents, buffers, nucleotides, modified nucleotides, reagents for additional assays.

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Tethering the 5′ End of RNA to a Three-Dimensional Matrix

This example describes the immobilization of the RNA contained in formalin-fixed paraffin-embedded (FFPE) mouse brain (mBrain) tissue samples and subsequent embedding of the FFPE mBrain samples in a hydrogel matrix for imaging. FFPE tissue samples can contain degraded RNA due to the storage and/or processing conditions, including baking, deparaffinization, decrosslinking and permeabilization that these samples undergo in order to facilitate analysis and imaging. In some cases, a significant amount of fragmented RNA can be lost during sample processing such as during decrosslinking of the sample, thus limiting the amount of RNA material present for imaging.

Sample Preparation: First, FFPE mBrain samples were baked at 60° C. for 2 hr and deparaffinized via (a) xylene treatment-incubation in 100% xylene twice for (2×) 10 min each, followed by (b) serial ethanol treatment-incubation with 100% ethanol (2×) 3 min each, then 96% ethanol 2× 3 min each, then 70% ethanol once (1×) 3 min, followed by brief immersion in nuclease-free water (lx 20 sec). The FFPE mBrain samples were not de-crosslinked or permeabilized.

5′ tethering of the RNA within the tissue sample was accomplished according to one of three protocols described below.

### 1. Enzymatic 5′ Tethering

The samples were treated with T4 polynucleotide kinase (T4 PNK) for 30 minutes at 37° C. to polish the ends of RNA fragments lacking 5′ phosphates. As shown in FIG. 1, samples were incubated with T4 RNA Ligase 1 and tethering oligo (20 nM) with PEG8000 (25%) at 25° C. for 4 hours. Samples were then incubated with a monomer buffer and polymerization mixture (4% acrylamide, 0.2% Bis, 0.2% ammonium persulfate, 0.2% TEMED), decrosslinked, and ribosomes were removed using 200 μg/ml Proteinase K, and 1% SDS in PBS (pH 8.5) at 50° C. for 3 minutes.

### 2. Organic 5′ Tethering Using Pyridine

The samples were treated with T4 polynucleotide kinase (T4 PNK) for 30 minutes at 37° C. to polish the ends of RNA fragments lacking 5′ phosphates. As shown in FIG. 2A, samples were incubated in 100 mM Carbonyldiimidizol in anhydrous Dimethyl formamide for 2 hours at RT followed by a 10 minute 20° C. incubation in Pyridine. The samples were then incubated with 2-aminoethyl methacrylamide (2-AEM) in H₂O to generate hydrogel reactive RNA molecules. Samples were then incubated with a monomer buffer and polymerization mixture (4% acrylamide, 0.2% Bis, 0.2% ammonium persulfate, 0.2% TEMED), decrosslinked and ribosomes were removed using 200 μg/ml Proteinase K, and 1% SDS in PBS (pH 8.5) at 50° C. for 3 minutes.

### 3. Organic 5′ Tethering Without Pyridine

The samples were treated with T4 polynucleotide kinase (T4 PNK) for 30 minutes at 37° C. to polish the ends of RNA fragments lacking 5′ phosphates. As shown in FIG. 2A, samples were incubated in 100 mM Carbonyldiimidizol in anhydrous Dimethyl formamide for 2 hours at RT and then washed 3 times in MeOH followed by a 30-minute 50° C. incubation in 100 mM 2-AEM, 30 mM MgCl₂, 100 mM Triethylamine, 1 nM Aniline and RNAse inhibitor.

Samples were then incubated with a monomer buffer and polymerization mixture (4% acrylamide, 0.2% Bis, 0.2% ammonium persulfate, 0.2% TEMED), decrosslinked and ribosomes were removed using 200 μg/ml Proteinase K, and 1% SDS in PBS (pH 8.5) at 50° C. for 3 minutes.

The embedded samples were contacted with a panel of probes targeting 50 mouse brain target genes, and reagents for ligation and amplification. Eight cycles of detection were performed to the barcode sequences associated with the probes. For a cycle of detection, a pool of intermediate probes targeting RCPs of the different genes were added and incubated, and fluorescently detected oligonucleotides were added to detect the intermediate probes. Different pools of intermediate probes were cycled and a universal pool of fluorescently labeled oligonucleotides were used to detect the intermediate probes in each cycle.

Through the sequential hybridization and detection cycles, fluorescent signals from the RCPs were detected and recorded at locations in the biological sample. The order of signals (or absence thereof) at a given location through the multiple cycles provided a signal code sequence for the RCP at the location, and the signal code sequence was compared to those in the codebook to identify a corresponding barcode sequence in the RCP and the gene associated therewith.

Images of the samples showed numerous identifiable RCP puncta in all fluorescent channels, thereby confirming that RNA were successfully immobilized into the FFPE mBrain samples prior to embedding of the tissue samples in a hydrogel matrix and subsequent analytical work-up.

### Example 2: In Situ Analysis Tissue Samples Containing 5′ Tethered RNA

This example describes results from the in situ analysis of formalin-fixed paraffin-embedded (FFPE) mouse brain (mBrain) tissue samples wherein the 5′ ends of RNA molecules within the tissue samples were tethered during sample preparation according to the methods disclosed in Example 1.

FFPE mBrain samples were baked at 60° C. for 2 hr and deparaffinized via (a) xylene treatment—incubation in 100% xylene twice for (2×) 10 min each, followed by (b) serial ethanol treatment—incubation with 100% ethanol (2×) 3 min each, then 96% ethanol 2× 3 min each, then 70% ethanol once (1×) 3 min, followed by brief immersion in nuclease-free water (1× 20 sec).

As a control, tissue samples were processed without the tethering of RNA molecules within the sample and no hydrogel embedding was performed. Two separate experiments with two control samples each were performed.

A second experimental group comprised tissue samples in which RNA molecules were enzymatically tethered via their 5′ ends according to the protocol exemplified in FIG. 1 and Example 1.

A third experimental group comprised tissue samples in which RNA molecules were enzymatically tethered via both the 5′ and 3′ ends of the RNA. To accomplish this method of tethering, tissue samples were incubated with T4 PNK to polish the 3′ ends into diols and add 5′ phosphates to the RNAs, then incubated with T4 RNA Ligase 1 and 20 nM tethering oligo with 25% PEG8000 at 25° C. for 2h followed directly by a 3′ RNA tethering protocol including RNA oxidation with 20 mM NaIO₄, aldehyde coupling (with 2AEM, methacrylamide, Et₃N, and aniline), and imine reduction with 0.2M NaBH₄. The tissue samples were then incubated with a monomer buffer and polymerization mixture (4% acrylamide, 0.2% Bis, 0.2% ammonium persulfate, 0.2% TEMED), de-crosslinked and ribosomes were removed using 200 μg/ml PK at 50° C. for 3 minutes.

Tissue samples from these three experimental groups were then embedded according to the protocol described in Example 1. The embedded samples were contacted with a panel of probes targeting 50 different genes expressed in the mouse brain. The tissue samples were then processed and analytes were detected according to the protocol described in Example 1 and imaged on a Zeiss Lapis fluorescence microscope.

As shown in FIG. 4A, 5′ enzymatic tethering of RNA molecules resulted in significant improvements in analyte detection compared to control tissue sections. As shown in FIGS. 4B-4E, the number of detected blobs observed was significantly higher in tissue samples in which RNA molecules were tethered via their 5′ ends. Number of detected blobs was quantified for four fluorescent dyes detected with the green channel (FIG. 4B), with the yellow channel (FIG. 4C), with the orange channel (FIG. 4D), and with the red channel (FIG. 4E). In addition, the number of decoded transcripts per 100 μm² was at least 20% greater in 5′ tethered samples compared to control samples (FIG. 4F). Moreover, the density of detected objects per μm² in 5′ tethered samples was about double that of the best control sample (FIG. 4G). As shown in FIG. 4H, the number of genes detected per cell was improved in 5′ tethered samples compared to control samples. In addition, the stripping error rate showed that some fluorescently detected oligonucleotides were not removed in 5′ tethered samples but the observed rates were within acceptable levels for the assay (FIG. 4I). Some tissue detachment was also observed with the control sample. Together these results suggest that 5′ tethering of RNA molecules during sample preparation leads to significant improvements in in situ analysis of tissue samples.

### Example 3: Polishing and Repair of 3′ and 5′ RNA Fragments in a Fixed Biological Sample

This example describes a workflow for repairing fragmented mRNA in a fixed biological sample, for example, a formalin-fixed, paraffin-embedded (FFPE) tissue section. A workflow for repairing RNA in a fixed biological sample is shown, for example, in FIG. 8.

A formalin-fixed, paraffin-embedded (FFPE) tissue section is deparaffinized. Following deparaffinization, the tissue sample is contacted with a T4 polynucleotide kinase (T4 PNK) enzyme, under conditions such that 3′ ends of RNA fragments (such as 3′ phosphate or 2′3′ cyclophosphate fragments) and 5′ ends of RNA fragments (such as 5′ hydroxyl fragments) are polished by the T4 PNK to yield polished RNA fragments comprising a 3′ hydroxyl and a 5′ phosphate. The T4 PNK is provided in a buffer that comprises 10 mM MgCl₂, and incubated with the sample for at least 20 minutes at 37 C. Additional reaction conditions for 3′ and 5′ reactions are described, for example, in Wang LK and Shuman S, Mutational analysis defines the 5′-kinase and 3′-phosphatase active sites of T4 polynucleotide kinase, Nucleic Acids Research (2002); 30(4):1073-1080, the content of which is herein incorporated by reference in its entirety.

In some cases for repairing damaged bases, the tissue section is then contacted with one or more organocatalysts comprising a phosphanilate and/or an anthranilate, such that the one or more organocatalysts repair one or more nucleotide base adducts in the biological sample. Following adduct repair with organocatalysts, the tissue section is subsequently contacted with a glycosylase enzyme for excising base adducts that were not repaired via organocatalysis.

Following repair and excision of RNA base adducts in the tissue section, the tissue section is then contacted with a T4 RNA ligase 1, such that repaired and enzymatically polished 3′ and 5′ ends of RNA fragments are ligated together to form a repaired RNA molecule.

Repaired and ligated RNA molecules may be processed further and/or used for various downstream assays. For example, following ligation of the polished RNA fragments to generate repaired RNA molecules, the tissue sample is contacted with a reverse transcriptase with a translesion or promiscuous reverse transcriptase such as HIV-1-RT, thus allowing for the creation of cDNA libraries from repaired RNA molecules in the tissue section. For in situ detection, the repaired RNA fragments are tethered and immobilized within the tissue section, for example, as described in the preceding examples. In other cases, the repaired and ligated RNA molecules or products thereof are released from the tissue sample for single cell barcoding and sequencing, or migrated to and captured on a spatial array for downstream analysis (e.g., sequencing).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-145. (canceled)

146. A method for analyzing a biological sample, comprising:
(a) providing a biological sample comprising a ribonucleic acid (RNA) comprising a 5′-phosphate group or a 5′-phosphate group modified with a leaving group;
(b) contacting the biological sample with an attachment agent, wherein the attachment agent comprises:
(i) at least one reactive moiety capable of reacting with at least one 5′-phosphate group of the RNA or at least one 5′-phosphate group of the RNA modified with a leaving group, and
(ii) at least one attachment moiety capable of attaching covalently or noncovalently to a matrix-forming agent;
(c) forming a covalent bond between the reactive moiety of the attachment agent and the RNA;
(d) contacting the biological sample with a matrix forming agent; and
(e) forming a three-dimensional polymerized matrix from the matrix-forming agent, thereby embedding the biological sample and immobilizing the RNA in the three-dimensional polymerized matrix.

147. The method of claim 146, wherein before the providing in (a), the method further comprises reacting at least one RNA in the biological sample with a polynucleotide kinase to provide the RNA comprising a 5′-phosphate group.

148. The method of claim 147, further comprising modifying the 5′-phosphate group with a leaving group to provide the RNA comprising a 5′-phosphate group modified with a leaving group.

149. The method of claim 147, wherein the polynucleotide kinase is a T4 Polynucleotide Kinase (T4 PNK) or a T7 Polynucleotide Kinase (T7-PNK).

150. The method of claim 146, wherein the attachment agent is a compound of Formula 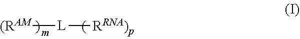
or a salt thereof, wherein:
each R^{RNA} is independently a reactive moiety capable of reacting with the at least one 5′-phosphate group of the RNA or the at least one 5′-phosphate group of the RNA modified with a leaving group;
each R^{AM} is independently an attachment moiety capable of attaching covalently or noncovalently to a matrix-forming agent;
L is a bond or a linker moiety;
m is an integer from 1 to 4; and
p is an integer from 1 to 4.

151. The method of claim 146, wherein the at least one reactive moiety of the attachment agent is capable of reacting with the at least one 5′-phosphate group of the RNA.

152. The method of claim 146, wherein the leaving group is any one of Cl, Br, I, —OR, —OC(O)R, —OS(O)₂R, or —NR¹R²,
wherein each R is independently haloalkyl, phenyl substituted with one or more alkyl or haloalkyl, or heteroaryl substituted with one or more alkyl or haloalkyl, and
wherein R¹ is independently H, alkyl, or haloalkyl, R² is independently haloalkyl, phenyl substituted with one or more alkyl or haloalkyl, or heteroaryl substituted with one or more alkyl or haloalkyl, or R¹ and R² are taken together with the N atom to which they are attached to form a heteroaryl.

153. The method of claim 146, wherein the forming in (c) comprises forming a covalent bond between the reactive moiety of the attachment agent and a 5′-phosphate group of the RNA without catalysis of an enzyme.

154. The method of claim 146, wherein the reactive moiety comprises or is a nucleophilic group.

155. The method of claim 146, wherein each attachment moiety of the at least one attachment moiety is independently or independently comprises a phenol moiety, an alkyne moiety, a norbornene moiety, a sulfide moiety, a furan moiety, a maleimide moiety, or an allyl ester moiety.

156. The method of claim 146, wherein the at least one attachment moiety is or comprises a moiety of Formula (I-b) 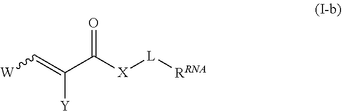
wherein
R^{RNA} is independently a reactive moiety capable of reacting with the at least one 5′-phosphate group of the RNA or the at least one 5′-phosphate group of the RNA modified with a leaving group;
L is a bond or a linker moiety;
W is independently H or C₁₋₆ alkyl;
Y is H or C₁₋₆ alkyl; and
X is NH, N(C₁₋₆ alkyl), or O.

157. The method of claim 146, further comprising, after the forming in (e), contacting the biological sample with a probe or probe set that binds directly or indirectly to the ribonucleic acid.

158. The method of claim 157, wherein the probe or probe set is a circular or circularizable probe or probe set, and the method comprises circularizing the circularizable probe or probe set using the ribonucleic acid or a product thereof as a template and generating an RCA product using the circular or circularizable probe as a template.

159. The method of claim 157, comprising imaging the biological sample to detect the probe or probe set or the RCA product.

160. The method of claim 157, wherein the probe or probe set comprises a barcode sequence.

161. The method of claim 160, wherein the method comprises detecting the barcode sequence or a complement thereof in the probe or probe set or in a product of the probe or probe set.

162. The method of claim 146, further comprising treating the biological sample with a detergent and a protease after forming the three-dimensional polymerized matrix in (e).

163. The method of claim 159 wherein the detecting comprises sequential hybridization and detection with a plurality of labelled probes.

164. The method of claim 159 wherein the detecting comprises performing sequencing-by-synthesis (SBS), sequencing-by-avidity (SBA) or sequencing-by-binding (SBB).

165. The method of claim 164, wherein the biological sample is contacted with a sequencing primer and a cyclic series of nucleotide incorporation or binding is performed.
